(19) United States
(12) Patent Application Publication
(10) Pub. No.: US 2026/0345012 A1
(43) Pub. Date: Nov. 5, 2026

(54) PHOTOACOUSTIC IMAGING FOR INTRAOPERATIVE EVALUATION AND TREATMENT OF PERIPHERAL NERVE INJURIES

(71) Applicant: THE JOHNS HOPKINS UNIVERSITY, Baltimore, MD (US)

(72) Inventors: Muyinatu BELL, Baltimore, MD (US); Michelle GRAHAM, Baltimore, MD (US); Nicholas VON GUIONNEAU, Baltimore, MD (US); Sami TUFFAHA, Baltimore, MD (US)

(21) Appl. No.: 19/118,094

(22) PCT Filed: Oct. 2, 2023
(86) PCT No.: PCT/US2023/034253

§ 371 Date: Apr. 3, 2025

Related U.S. Application Data
(60) Provisional application No. 63/379,005, filed on Oct. 11, 2022.

(51) Int. Cl. A61B 5/00; G06T 7/00; G06T 7/38

(52) U.S. Cl. CPC A61B 5/0095; A61B 5/4041; A61B 5/7264; G06T 7/0012; G06T 7/38; A61B 2576/02; G06T 2207/10132; G06T 2207/30004

(57) ABSTRACT

A system for assessing a nerve in a patient includes an encasement configured to be positioned at least partially around the nerve. The system also includes one or more fibers coupled to the encasement. Light is transmitted through the one or more fibers, through the encasement, and onto the nerve. A sound with ultrasonic MHz frequencies is generated in response to the nerve absorbing the light. The system also includes an ultrasound transducer coupled to the encasement, the one or more fibers, or both. The ultrasound transducer is configured to measure the sound.

### CROSS-REFERENCE TO RELATED APPLICATIONS

This is the national stage entry of International Patent Application No. PCT/US2023/034253, filed on Oct. 2, 2023, and published as WO 2024/081119 A1 on Apr. 18, 2024, which claims the benefit of U.S. Provisional Patent Application No. 63/379,005, filed on Oct. 11, 2022, which are hereby incorporated by reference in their entireties.

### GOVERNMENT SUPPORT

This invention was made with government support under grant 2014088 and ECCS 1751522 awarded by the National Science Foundation. The government has certain rights in the invention.

### FIELD OF THE DISCLOSURE

The present disclosure relates generally to systems and methods for the evaluation and/or treatment of nerve injuries. More particularly, the present disclosure relates to systems and methods for interoperative evaluation and/or treatment of peripheral nerve injuries using photoacoustic imaging.

### BACKGROUND OF THE DISCLOSURE

The peripheral nervous system is a network of motor and sensory nerves that connect the brain and spinal cord (e.g., the central nervous system) to the entire human body. These nerves control the functions of sensation, movement, and motor coordination. They are delicate structures and can be damaged easily. People with traumatic nerve damage can experience severe, unrelenting pain, burning sensation, tingling, numbness, and/or paralysis in the part of the body affected by the damaged nerve.

A classification system called the Sunderland classification system defines five different degrees of peripheral nerve injury. The first degree includes a reversible local conduction block at the site of the injury. This injury does not require surgical intervention and usually will recover within a matter of hours to a few weeks. The second degree involves a loss of continuity of the axons (e.g., the “electrical wires”) within the nerve while the nerve architecture remains intact. Surgical intervention is usually not required for this type of injury as spontaneous axonal regeneration and motor/sensory recovery will occur. The third degree includes damage to the axons and their supporting structures within the nerve. In this case, spontaneous recovery may or may not occur. The fourth degree includes damage to the axons and the surrounding tissues sufficient to create scarring that prevents nerve regeneration. Surgical intervention with nerve grafting is necessary to repair the injury. The fifth degree is usually found in laceration or severe stretch injuries. The nerve is divided into two. The only way to repair a fifth-degree injury is through surgery.

Because the available diagnostic modalities are not able to accurately and reliably identify which nerve injuries will spontaneously recover and which will not, most nerve injuries are initially managed with a period of observation, during which serial exams and electrodiagnostic testing are employed to evaluate for signs of early recovery. If recovery is not observed 3-6 months after the injury, surgical exploration is typically indicated. During this observation period, progressive irreversible atrophy of the affected muscle lacking nerve supply takes place, downgrading the potential for functional recovery with surgical intervention. However, the observation period is needed to avoid many unnecessary surgeries. During surgical exploration, the affected nerves are electrically stimulated to evaluate for muscle contractions. If muscle contractions are noted, surgical nerve repair is typically not performed. If no muscle contractions are observed, nerve repair may be employed with nerve grafts or distal nerve transfers. However, the lack of muscle contraction does not rule out the possibility that nerve regeneration is progressing towards the affected muscle without having reached it. There are no diagnostic modalities available to identify the presence and location of regenerating axons within nerve tissue. Therefore, nerve repairs are sometimes performed unnecessarily. Furthermore, it is often difficult to know where along the length of a nerve to intervene.

There are several unmet needs in peripheral nerve surgery: (1) how to precisely determine the location and extent of a nerve injury, (2) whether nerve regeneration is proceeding beyond the injury site and to what location along the length of the nerve regeneration has proceeded and (3) how to monitor nerve regeneration postoperatively. For example, surgeons currently use crude, rudimentary techniques to estimate the location and extent of a nerve injury. Most commonly, they palpate a nerve in the hope of finding evidence of an injury manifesting as firmness from scarring. They may also practice “bread loafing” where they serially trim back the proximal nerve segment until they think they see nerve tissue that grossly appears “healthy”-both approaches are highly subjective and prone to error. Electrical stimulation of the nerve intraoperatively is of limited value as it can only ascertain whether or not axons are innervating muscle. Imaging modalities like MRI and ultrasound can only provide information pertaining to nerve architecture and do not allow for visualization of the axons within nerve tissue. Diffusion tensor imaging (DTI) was touted as a promising solution but failed in clinical translation due to limited resolution and difficulties with motion artifact. Therefore, what is needed is a system and method for solving these unmet needs.

### SUMMARY

A system for assessing a nerve in a patient is disclosed. The system includes an encasement configured to be positioned at least partially around the nerve. The system also includes one or more fibers coupled to the encasement. Light is transmitted through the one or more fibers, through the encasement, and onto the nerve. A sound with MHz ultrasonic frequencies (i.e., outside of the range of human hearing) is generated in response to the nerve absorbing the light. The system also includes an ultrasound transducer coupled to the encasement, the one or more fibers, or both. The ultrasound transducer is configured to measure the sound.

A method for assessing a nerve in a patient is also disclosed. The method includes creating an incision in the patient to expose the nerve within the patient. The method also includes positioning an encasement at least partially around the nerve. The method also includes transmitting light through the encasement to the nerve. A first portion of the light shines on a first portion of the nerve. A second portion of the light shines a second portion of the nerve. A sound (with MHz ultrasonic frequencies) is generated in response to the nerve absorbing the light. The method also includes measuring the sound using an ultrasound transducer. The method also includes generating a photoacoustic image based at least partially upon the sound.

In another embodiment, the method includes creating an incision in the patient to expose the nerve within the patient. The method also includes positioning an encasement at least partially around the nerve. The encasement includes first and second encasement portions that are configured to actuate between an open position and a closed position. The first and second encasement portions in the closed position are configured to secure the nerve within a channel defined by inner surfaces of the first and second encasement portions. The method also includes moving the encasement along the nerve from a first location to a second location. The method also includes transmitting light through the encasement to the nerve. The light is transmitted through the encasement when the encasement is at the first location and the second location. A first portion of the light is transmitted through the first encasement portion onto a first circumferential portion of the nerve. A second portion of the light is transmitted through the second encasement portion onto a second circumferential portion of the nerve. The light includes different wavelengths. A number of the different wavelengths is from about 2 to about 50. The different wavelengths range from about 690 nm to about 2200 nm. A sound (with MHz ultrasonic frequencies) is generated in response to the nerve absorbing the light. The nerve absorbs different amounts of the light at the different wavelengths. An amplitude of the sound depends at least partially upon the different amounts of the light that are absorbed. The method also includes measuring the amplitude of the sound using an ultrasound transducer. The ultrasound transducer is coupled to the encasement. The method also includes generating a photoacoustic image based at least partially upon the amplitude of the sound. The method also includes determining a spectral response based at least partially upon the photoacoustic image. The spectral response comprises the amplitude of the sound at the different wavelengths of the light. The spectral response being greater than a spectral response threshold indicates an amount of myelin in the nerve is greater than a predetermined myelin threshold. The spectral response being less than the spectral response threshold indicates the amount of the myelin is less than the predetermined myelin threshold. The method also includes determining a nerve condition of the nerve based at least partially upon the spectral response. The nerve condition is determined at the first location, the second location, or both.

### BRIEF DESCRIPTION OF THE FIGURES

FIGS. 1 and 2 illustrate perspective views of a system for assessing a nerve in a patient, according to an embodiment.

FIG. 3 illustrates a front view of an inner surface of a portion of an encasement of the system, according to an embodiment.

FIG. 4 illustrates a flowchart for a method for assessing the nerve in the patient, according to an embodiment.

FIG. 5 illustrates an overlaid image, according to an embodiment.

FIG. 6 illustrates a photoacoustic image, according to an embodiment.

FIG. 7 illustrates an image (e.g., the overlaid image) including a region of interest, according to an embodiment.

FIGS. 8A and 8B illustrate graphs showing spectral responses, according to an embodiment.

FIGS. 9A-9C illustrate the results of the multispectral nerve classification algorithm, according to an embodiment.

FIG. 10 illustrates an experimental setup for in vivo photoacoustic imaging, including bifurcated fiber bundles secured in a custom-molded agarose block to bilaterally illuminate the nerve, with an ultrasound transducer positioned to image a circular cross section of the illuminated nerve, according to an embodiment.

FIGS. 11A-11C illustrate graphs showing spectroscopic optical absorbance measurements of five samples (i.e., PBS, 3% w/v agarose, cholesterol, one median nerve, and the average±one standard deviation of three ulnar nerve samples), according to an embodiment.

FIGS. 12A-12D illustrate images showing co-registered photoacoustic and ultrasound images of swine ulnar and median nerves when bilaterally illuminated with an optical wavelength of 1725 nm, according to an embodiment.

FIGS. 13A-13D illustrate measured photoacoustic amplitude spectra of each pixel within the ROIs shown in FIGS. 12C and 12D, according to an embodiment.

FIGS. 14A and 14B illustrate graphs that directly compare the mean±one standard deviation of the photoacoustic amplitude spectra residing within the horizontal dashed lines in FIG. 13A-13D and the corresponding absorbance spectra replicated from FIGS. 11A-11C, according to an embodiment.

FIGS. 15A-15D illustrate graphs showing a comparison of the NIR-II and NIR-III photoacoustic amplitude spectra for the in vivo swine median and ulnar nerves and surrounding agarose, according to an embodiment.

FIGS. 16A-16H illustrate representative histological sections of swine ulnar and median nerve samples, according to an embodiment.

FIGS. 17A-17D illustrate graphs showing normalized subtraction of spectrophotometer measurements, according to an embodiment.

### DETAILED DESCRIPTION

Intraoperative monitoring of nerves may facilitate safe and successful surgery across a wide variety of disciplines. For example, in neurosurgery, real-time detection of nerves can prevent iatrogenic complications such as blindness and paralysis. Additionally, in nerve repair surgery, real-time analysis of the extent of nerve injury may be used to regain normal muscular function postoperatively. However, as described above, current intraoperative techniques utilize ionizing radiation, are highly subjective, have insufficient contrast, and/or are difficult to integrate into the surgical workflow.

Photoacoustic imaging is an intraoperative technique for high-contrast, real-time monitoring of nerve tissue without utilizing ionizing radiation. This imaging technique relies on the photoacoustic effect in which tissues selectively absorb incident laser light based on their inherent optical properties. The subsequent local heating emits acoustic waves (e.g., sound) which are sensed (e.g., measured) by an ultrasound receiver. However, photoacoustic imaging of nerves is challenged by competing chromophores in the field of view as well as insufficient optical penetration into nerve tissue, which produces minimal photoacoustic signal generation (and in the worst cases, no photoacoustic signals are produced).

In biological tissues, many chromophores can emit photoacoustic signals, including water, hemoglobin in blood, lipids, collagen, and melanin. Although the optical absorption spectrum of each chromophore is unique, there are many wavelengths at which the optical absorption of these chromophores overlaps, or the chromophore of interest absorbs less than background chromophores. This can lead to the photoacoustic signal from nerves being overpowered by more strongly absorbing chromophores or difficulty in differentiating nerve tissue from surrounding tissues with similar absorption at a particular wavelength. Therefore, a dual-wavelength approach may be used to selectively amplify the photoacoustic signals from blood vessels and nerves using, for example, 750 nm and 1230 nm wavelengths.

The system and method described herein include a (e.g., handheld) photoacoustic imaging device and an image processing algorithm to intraoperatively localize and evaluate the extent of a peripheral nerve injury. The system and method may virtually biopsy, and thus quantitatively assess, viable nerve tissues. The system and method may also quantify peripheral nerve viability intraoperatively and monitor nerve regeneration prior to target organ reinnervation. The system and method may also provide surgeons with an objective measure of nerve viability. As a result, the system and method will transform the clinical decision-making paradigm in peripheral nerve injury surgery from best-guess practice into a highly reliable data-driven approach with improved patient outcomes.

The system and method described herein may accomplish the foregoing by generating deeply penetrating optical illumination (e.g., light). The high optical scattering coefficient of nerve tissues reduces light penetration into the nerve, and thereby reduces the strength of the photoacoustic signal. Therefore, the light delivery device may optimize the light penetration into the nerve tissue by exploiting multiple light trajectories. Simulations of this optimization have been performed for the creation of scenario-specific deeply penetrating optical illumination (e.g., for nerves of differing diameters or composition).

Photoacoustic imaging of a nerve presents multiple challenges. For example, tissue shift from breathing or surgical manipulation introduces motion artifacts in photoacoustic images which cause unreliable or irreproducible measurements. In addition, successful implementation of deeply penetrating optical illumination involves a custom illumination profile. Lastly, robust use of photoacoustic imaging requires standardization of the relative relationships of the nerve and optical devices.

Therefore, the handheld nerve imaging device described herein secures the nerve, optical devices, and acoustic receivers in the optimal relative positions for the duration of the intraoperative imaging procedure. Because the device joins all components of the photoacoustic imaging system into a single unit, the device eliminates troublesome motion artifacts. The device also affixes the nerve in the intended position within the custom deeply penetrating illumination profile to achieve maximum optical penetration. In addition, the device standardizes the imaging configuration, enabling faster training of surgeons as well as reproducibility within and between patients.

The handheld device may be fabricated using a custom, 3D-printed mold and material that is both optically-transparent and acoustically-mimics nerve tissues (e.g., plastisol, agar). The device may include a flexible ring which opens and closes to encase the nerve and secure the nerve in place. The device can be repositioned multiple times during the surgery. The device may also be compatible with alternating, rotating, and/or flexible ultrasound receivers for multi-angle ultrasound reception for visualization of the entire nerve structure. Furthermore, although the device interfaces with sterilizable, non-disposable equipment (e.g., light source, optical fibers, ultrasound transducer, and ultrasound machine), the device may be disposable. The disposable nature of the device enables customization of the imaging configuration to fit the imaging scenario (e.g., nerves of differing diameters or composition).

The clinical user interface provided by the device may be used for multispectral photoacoustic imaging of nerves in the clinic. Clinical user interface may provide clinically meaningful quantitative thresholds pertaining to the amount of myelination present within the nerve to allow the surgeon to make critical surgical decisions. This user interface may also provide a simultaneously multi-wavelength image display. Overall, the user interface may enable smooth integration of photoacoustic imaging into the surgical workflow.

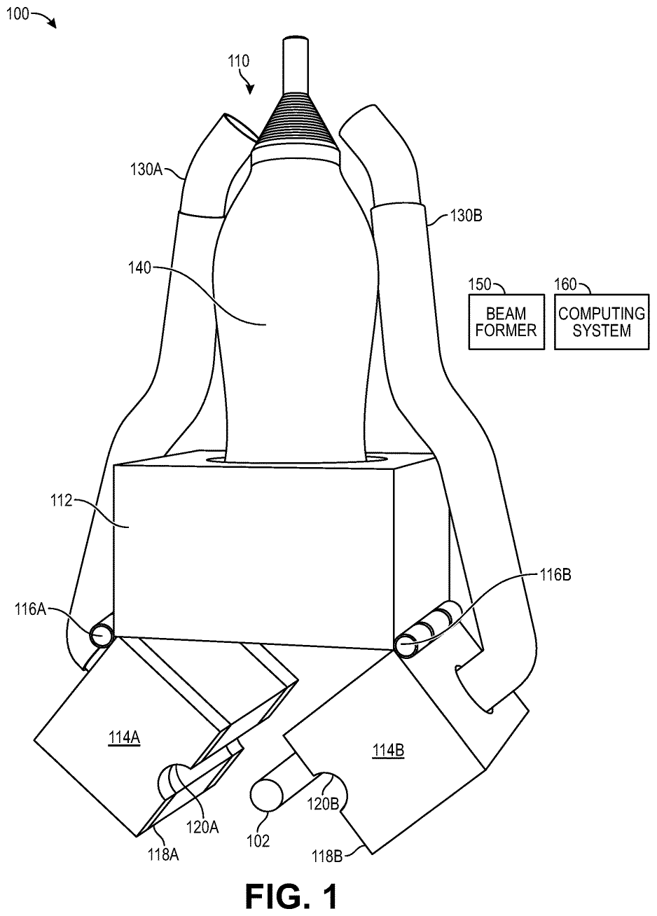

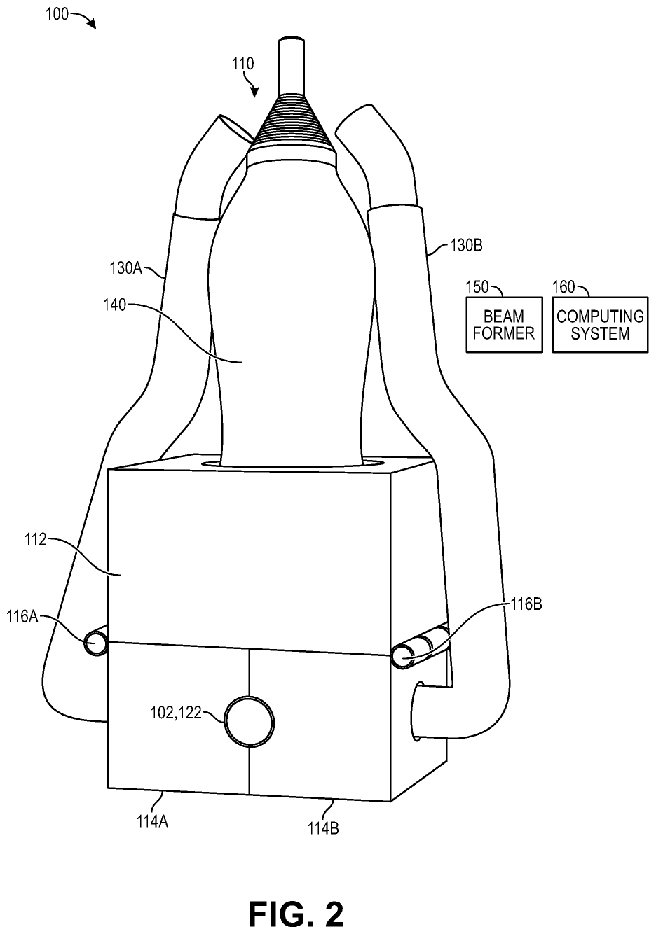

FIGS. 1 and 2 illustrate perspective views of a system 100 for evaluating a nerve 102 in a patient, according to an embodiment. More particularly, the system 100 may be or include a handheld device 110 that may intraoperatively evaluate and/or treat a peripheral nerve injury in the patient using photoacoustic imaging.

The system 100 (e.g., the handheld device 110) may include an encasement 112 that is configured to be positioned at least partially around a nerve 102 in the patient. The encasement 112 may include one or more encasement portions (two are shown: 114A, 114B). The encasement portions 114A, 114B may be configured to actuate (e.g., pivot) between an open position (FIG. 1) and a closed position (FIG. 2). In one example, the encasement portions 114A, 114B may actuate/pivot via hinges 116A, 116B.

Each encasement portion 114A, 114B may include an inner surface 118A, 118B. The inner surfaces 118A, 118B may at least partially face one another. A distance between the inner surfaces 118A, 118B may decrease as the encasement portions 114A, 114B actuate from the open position to the closed position. In one embodiment, the inner surfaces 118A, 118B may contact one another in the closed position. The inner surfaces 118A, 118B may have a recess 120A, 120B formed therein.

In the open position (as shown in FIG. 1), the encasement portions 114A, 114B may be spaced apart from one another such that the nerve 102 may be inserted between and/or removed from the encasement portions 114A, 114B. In the closed position (as shown in FIG. 2), the nerve 102 may be secured in a channel 122 defined by the recesses 120A, 120B.

The system 100 (e.g., the handheld device 110) may also include one or more fibers (two are shown: 130A, 130B). The fibers 130A, 130B may be or include flexible, bifurcated fiber bundles that are configured to transmit light therethrough. As shown, the fibers 130A, 130B may be coupled to the encasement portions 114A, 114B, respectively. The first fiber 130A may transmit light to the first encasement portion 114A, and the second fiber 130B may transmit light to the second encasement portion 114B.

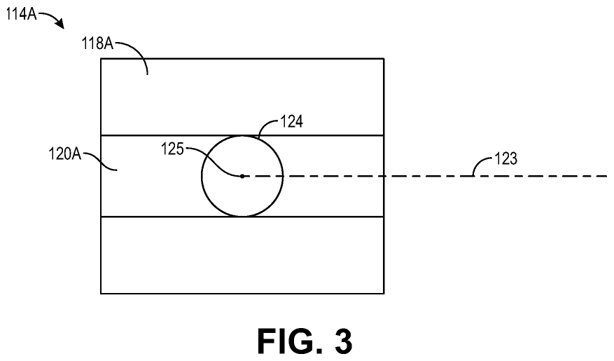

The light may travel through the encasement portions 114A, 114B and exit via one or more openings (only opening 124A is shown) in the inner surfaces 118A, 118B, as shown in FIG. 3. The opening 124A may intersect (e.g., extend through) the recess 120A. A central longitudinal axis 125 through the opening 124A may be substantially perpendicular to a central longitudinal axis 123 through the recess 120A and/or channel 122.

As a result, the light transmitted through the first fiber 130A and/or through the first encasement portion 114A may shine upon a first circumferential portion of the nerve 102, and the light transmitted through the second fiber 130B and/or through the second encasement portion 114B may shine upon a second circumferential portion of the nerve 102. The first and second circumferential portions may be circumferentially-offset from one another. For example, the first circumferential portion may include a first 180 degrees around the nerve 102, and the second circumferential portion may include the next/opposite 180 degrees around the nerve 102.

The system 100 (e.g., the handheld device 110) may also include an ultrasound transducer 140. The ultrasound transducer 140 may be coupled to the encasement 112, the fibers 130A, 130B, or a combination thereof. In the example shown, the ultrasound transducer 140 may be positioned above the encasement 112 (e.g., farther from the nerve 102 than the encasement 112). In the example shown, the ultrasound transducer 140 may be positioned between the fibers 130A, 130B. The ultrasound transducer 140 may be configured to measure a sound that is generated by light contacting the nerve 102 and/or the nerve 102 absorbing the light. In one embodiment, the ultrasound transducer 140 may also be configured to generate one or more photoacoustic images based at least partially upon the sound.

In at least one embodiment, the system 100 may also include one or more spatial coherence beamformers (e.g., a short-lag spatial coherence beamformer) 150. In one embodiment, the beamformer(s) 150 may be coupled to the handheld device 110. In another embodiment, the beamformer(s) 150 may be remote from the handheld device 110 and configured to communicate with the handheld device 110 through one or more wires or wirelessly. The beamformer(s) 150 may be configured to display the spatial coherence (rather than the amplitude) of the light and/or sound. The beamformer(s) 150 may also or instead be configured to create photoacoustic images that are presented as an overlay on ultrasound images, side-by-side with amplitude (e.g., ultrasound or photoacoustic) images, or independently of any other images.

The system 100 may also include a computing system 160. In one embodiment, the computing system 160 may be coupled to the handheld device 110. In another embodiment, the computing system 160 may be remote from the handheld device 110 and configured to communicate with the handheld device 110 through one or more wires or wirelessly. The computing system 160 may be used to implement one or more of the signal processing steps of the device (e.g., amplitude beamformer, spatial coherence beamformer, the photoacoustic spectra, objective measurement of nerve viability).

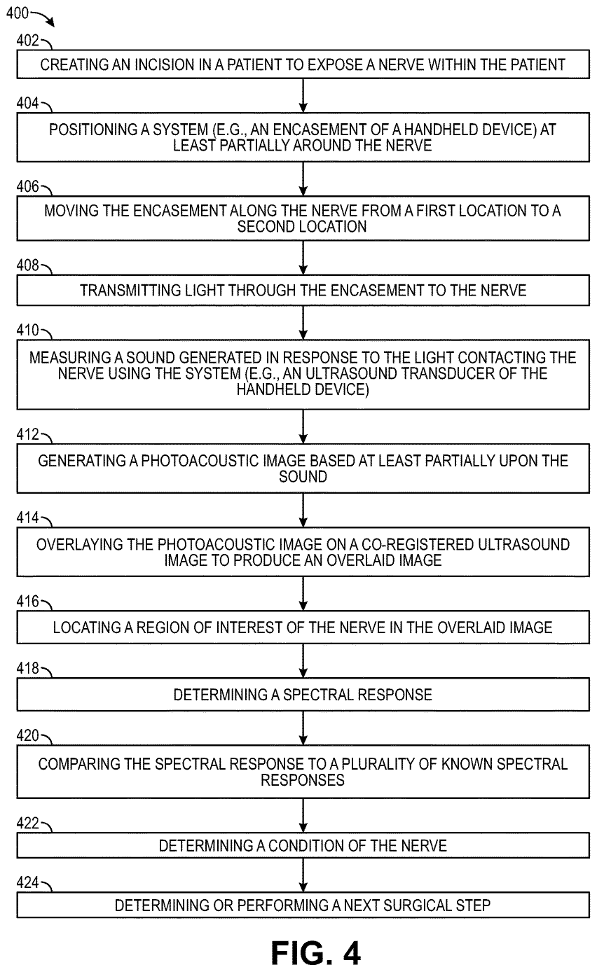

FIG. 4 illustrates a flowchart for a method 400 for assessing the nerve 102 in the patient, according to an embodiment. More particularly, the method 400 may be used to intraoperatively assess and/or treat a peripheral nerve injury using photoacoustic imaging. An illustrative order of the method 400 is provided below; however, one or more steps of the method 400 may be performed in a different order, combined, repeated, or omitted without departing from the scope of the disclosure.

The method 400 may include creating an incision in a patient to expose the nerve 102 within the patient, as at 402. The nerve includes myelin and axons. The myelin at least partially surrounds the axons. The myelin includes lipids.

The method 400 may also include positioning the system 100 at least partially around the nerve, as at 404. More particularly, this may include positioning the handheld device 110 (e.g., the portions 114A, 114B of the encasement 112) at least partially around the nerve 102. As described above, the first and second encasement portions 114A, 114B may be configured to actuate between an open position and a closed position. The first and second encasement portions 114A, 114B may be configured to move toward or away from the nerve 102 when in the open position. The first and second encasement portions 114A, 114B may be configured to secure the nerve 102 within the channel 122 when in the closed position.

The method 400 may also include moving the encasement 112 along the nerve 102 from a first location to a second location, as at 406. The first and second encasement portions 114A, 114B may be in the open position and/or the closed position while the encasement 114A, 114B moves.

The method 400 may also include transmitting light through the encasement 112 to the nerve 102, as at 408. The light may be transmitted through the encasement 112 when the encasement 112 is at the first location and/or the second location. A first portion of the light may be transmitted through the first encasement portion 114A onto a first circumferential portion of the nerve 102. A second portion of the light may be transmitted through the second encasement portion 114B onto a second circumferential portion of the nerve 102.

The light may have or include different wavelengths. In one example, the light may have a first wavelength at a first time, a second wavelength at a second time, and so on. In another example, the light may have multiple different wavelengths at the same time. A number of the different wavelengths may be from about 2 to about 50. The different wavelengths may range from about 690 nm to about 2200 nm or from about 1200 nm to about 1900 nm.

The nerve 102 may absorb at least a portion of the light. A sound (e.g., ultrasound waves) may be generated in response to the nerve 102 absorbing the light. The nerve (e.g., the lipids) may absorb different amounts of the light at the different wavelengths. An amplitude of the sound may depend at least partially upon the different amounts of the light that are absorbed.

The method 400 may also include measuring the sound using the ultrasound transducer 140, as at 410. The sound may be measured when the encasement 112 is transmitting the light at the first location and/or the second location.

The method 400 may also include generating a photoacoustic image based at least partially upon the sound, as at 412. The photoacoustic image may be generated by the handheld device 110 (e.g., the ultrasound transducer 140) or by the computing system 160. The photoacoustic image may be a spatial coherence photoacoustic image that is formed based at least partially upon the amplitude of the sound and/or the spatial coherence beamformer(s) 150.

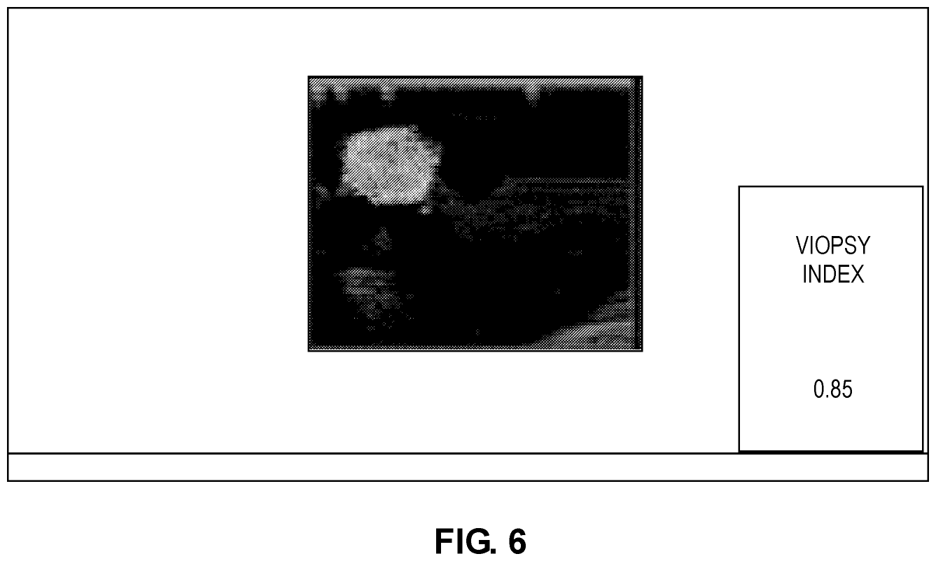

FIG. 6 illustrates a photoacoustic image, according to an embodiment. The image may show the axons within the nerve tissue, which may be used to make intraoperative decisions regarding whether surgical intervention is needed and/or precisely where along the nerve to intervene. The image may also or instead include a viopsy index.

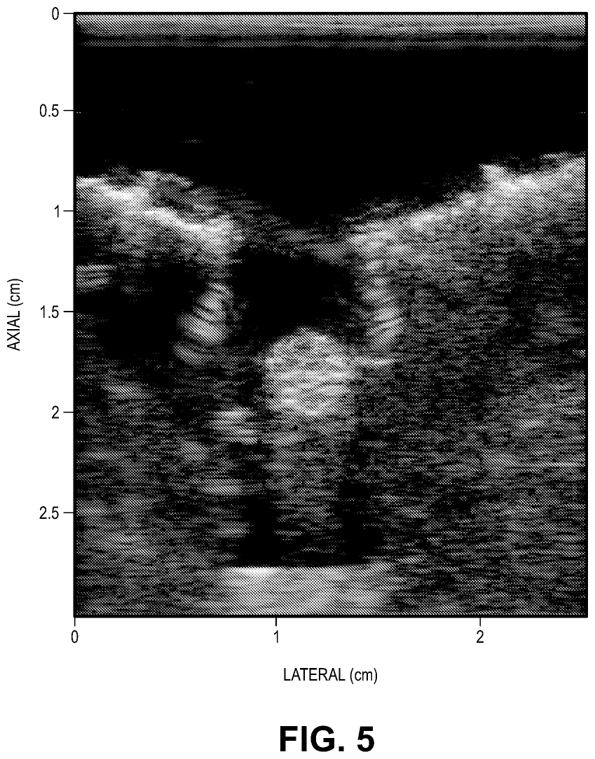

The method 400 may also include overlaying the photoacoustic image on a co-registered ultrasound image to produce an overlaid image, as at 414. FIG. 5 illustrates an overlaid image, according to an embodiment. The co-registered ultrasound image may be acquired with/using the system 100. For example, the co-registered ultrasound image may be acquired with/using the ultrasound transducer 140. The images may be overlaid using the handheld device 110 (e.g., the ultrasound transducer 140) or the computing system 160.

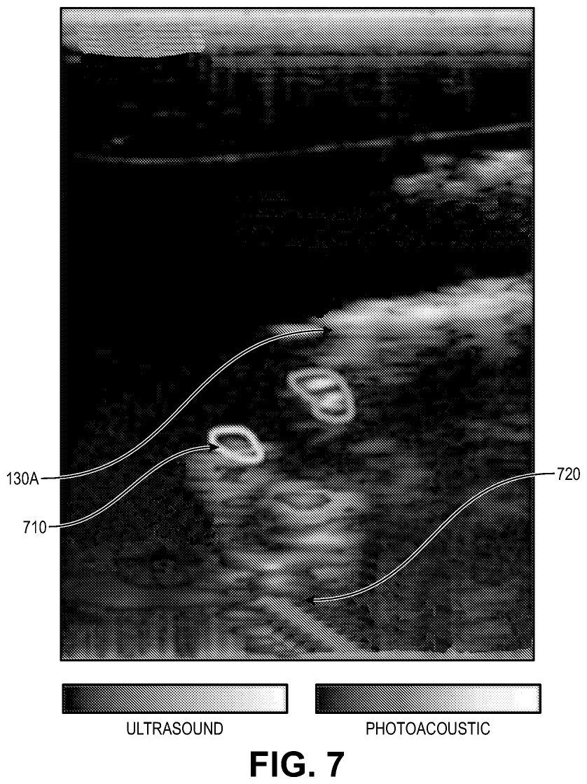

The method 400 may also include locating a region of interest on/in the nerve 102, as at 416. FIG. 7 illustrates an image including the region of interest 710, according to an embodiment. The region of interest 710 may be based at least partially upon (e.g., identified in) the photoacoustic image, the ultrasound image, the overlaid image, or a combination thereof. The region of interest 710 may be located using the handheld device 110 (e.g., the ultrasound transducer 140) or the computing system 160. The region of interest 710 may include a region of nerve tissue to be assessed, a region within surrounding background (e.g., non-nerve) tissue, and/or a region within the encasement material surrounding the nerve 102. This step may also or instead include locating photoacoustic signals from the fibers 130A and/or one or more acoustic reflection artifacts 720.

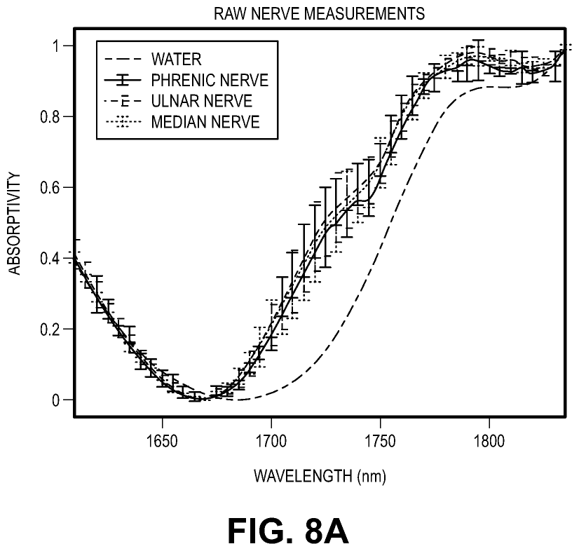

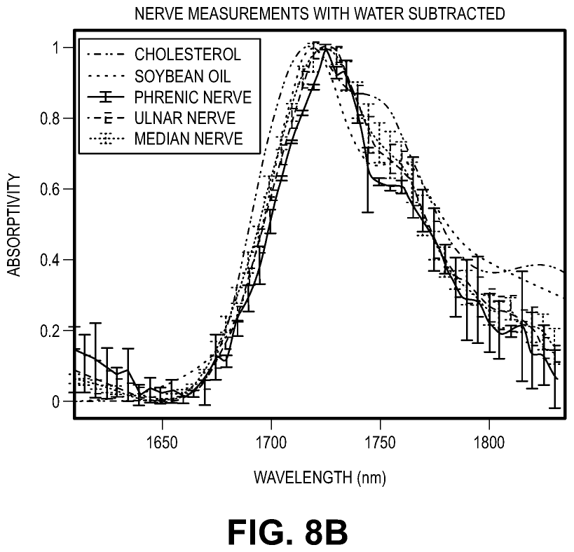

The method 400 may also include determining a spectral response, as at 418. FIGS. 8A-8C illustrate graphs showing spectral responses, according to an embodiment. More particularly, FIG. 8A shows raw nerve measurements with different curves for water, the phrenic nerve, the ulnar nerve, and the median nerve. FIG. 8B shows the nerve measurements with water subtracted. FIG. 8B includes different curves for cholesterol, soybean oil, the phrenic nerve, the ulnar nerve, and the median nerve. In FIGS. 8A and 8B, the X-axis represents wavelength (e.g., in nm), and the Y-axis represents absorptivity.

The spectral response may be based at least partially upon the sound, the photoacoustic image, the overlaid images, the region of interest, or a combination thereof. The spectral response may be determined using the handheld device 110 (e.g., the ultrasound transducer 140) or the computing system 160. The spectral response may include the amplitude of the sound at the different wavelengths of the light. The spectral response being greater than a spectral response threshold (e.g., at a selected wavelength) may indicate that an amount of the myelin is greater than a predetermined myelin threshold. In contrast, the spectral response being less than the spectral response threshold (e.g., at the selected wavelength) may indicate that the amount of the myelin is less than the predetermined myelin threshold.

The method 400 may also include comparing the spectral response to a plurality of known spectral responses, as at 420. The comparison may be performed by the handheld device 110 (e.g., the ultrasound transducer 140) or the computing system 160. The known spectral responses may be stored in a database. The database may also include a plurality of known nerve conditions that correspond to the known spectral responses. The known nerve conditions may include an uninjured (e.g., healthy) nerve condition and an injured (e.g., damaged) nerve condition. The injured (e.g., damaged) nerve condition may include a first state where regeneration is occurring at a specific site or sites along the length of the nerve 102 (e.g., in the region of interest), and/or a second state where no regeneration is occurring at a specific site or sites along the length of the nerve 102 (e.g., in the region of interest).

The method 400 may also include determining a nerve condition of the nerve 102, as at 422. The nerve condition may be determined using the handheld device 110 (e.g., the ultrasound transducer 140) or the comprising system 160. In one embodiment, the nerve condition may be based at least partially upon the absence, presence, and/or amount of myelin present (e.g., from step 418) as surrogate for myelinated axons. In another embodiment, the nerve condition may be based at least partially upon the comparison (e.g., from step 420). The nerve condition may be determined at the first location, the second location, or both.

The method 400 may also include determining or performing a next surgical step, as at 424. The next surgical step may be determined based at least partially upon the nerve condition (e.g., from step 422). The next surgical step may be or include to not cut (or stop cutting) the nerve 102, to determine where along the nerve 102 to intervene, to determine how far to resect the proximal nerve stump when reconstructing the nerve injury in discontinuity, to reconstruct the nerve 102 with a nerve graft, transfer, or both, or a combination thereof.

### Multispectral Nerve Classification Algorithm

Co-registered ultrasound and photoacoustic images were generated from channel data with delay-and-sum (DAS) and short-lag spatial coherence (SLSC) beamforming. Nerve and background regions of interest were selected by thresholding the normalized SLSC images with a threshold of 0.45. Regions were subsequently manually modified to remove artifacts and to separate the target and background regions of interest. The segmented regions were applied as a mask to DAS photoacoustic images. The amplitude of each pixel within the mask was measured as a function of wavelength to generate photoacoustic amplitude spectra.

This process was repeated for each experimental dataset (i.e., ulnar, proximal median, and distal median nerves). The photoacoustic amplitude spectra were divided into three classes: healthy nerve, experimental nerve, and background. A reference databank for each class was created by randomly sampling 80% of the photoacoustic amplitude spectra of each class. The remaining 20% of the photoacoustic amplitude spectra of each class was used as the class test set, mimicking what would be obtained from a region of interest within an intraoperative in vivo photoacoustic image of a nerve. For each class test set, a distribution of Pearson correlation coefficients, ρ_{c,i}(T_{q},D_{k,c}), was computed between the photoacoustic amplitude spectra of the testing set, T, and reference set, D, as described by the equations:

$${ρ}_{c , i} ( {T}_{q} , {D}_{k , c} ) = \frac{1}{( N - 1 )} {\sum }_{n = 1}^{N} ( \frac{{T}_{q , n} - {μ}_{{T}_{q}}}{{σ}_{{T}_{q}}} ) ( \frac{{D}_{k , c , n} - {μ}_{{D}_{k , c}}}{{σ}_{{D}_{k , c}}} ) \qquad ( 1 )$$

where

n=1 . . . N, c=1 . . . C, k=1 . . . K, i=1 . . . I, Q=1 . . . Q (2)

where n represents a sample (i.e., the photoacoustic signal at one wavelength), N represents the total number of samples in a single photoacoustic amplitude spectra, c represents the class, C represents the total number of classes, q represents a single photoacoustic amplitude spectra in the test set, Q represents the total number of photoacoustic amplitude spectra in the test set, k represents a single photoacoustic amplitude spectra in the reference databank, K represents the total number of photoacoustic amplitude spectra in reference databank, i represents the index of the pair of photoacoustic amplitude spectra being compared between the test and reference sets, and I represents the total number of unique pairwise photoacoustic amplitude spectra comparisons between the testing and reference datasets. The combinatoric rule of product computes I as I=K*Q.

A distribution of Pearson correlation coefficients was also computed between all unique pairwise combinations of photoacoustic amplitude spectra within the testing set, as described by:

$${\hat{ρ}}_{i} ( {T}_{q} , {T}_{k} ) = \frac{1}{N - 1} {\sum }_{n = 1}^{N} ( \frac{{T}_{q , n} - {μ}_{{T}_{q}}}{{σ}_{{T}_{0}}} ) ( \frac{{T}_{k , n} - {μ}_{{T}_{k}}}{{σ}_{{T}_{k}}} ) \qquad ( 3 )$$

where

$${}_{Q} {I}_{2} = \frac{Q !}{2 ! ( Q - 2 ) !} .$$

The medians of ρ_{c,i} were compared with the median of ρ\hat{ }. The class with the associated ρ_{c,i} whose median was most similar to ρ\hat{ } was determined as the classification of the testing set. This analysis was repeated for 100 random samplings of the data into the reference databank and testing sets to determine a classification accuracy.

### Results

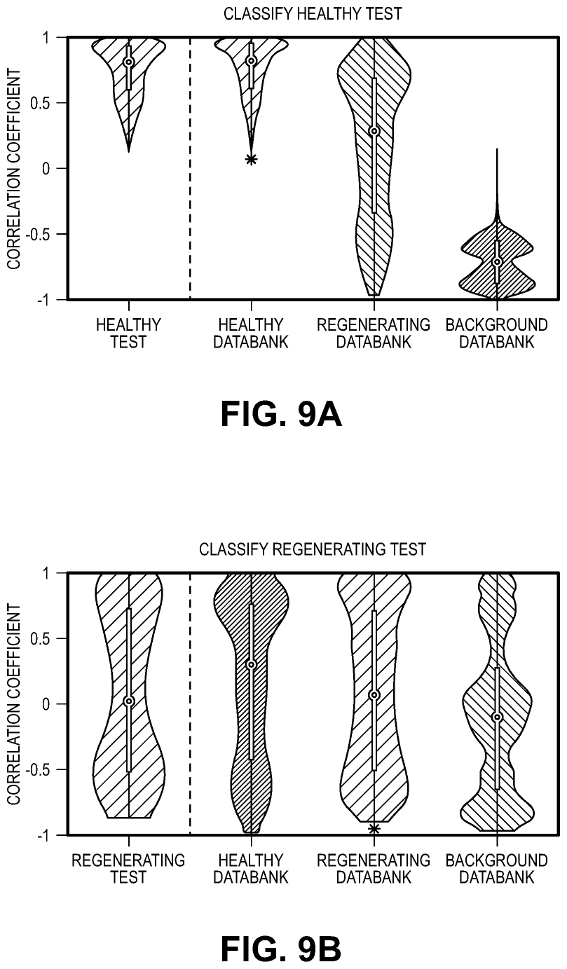

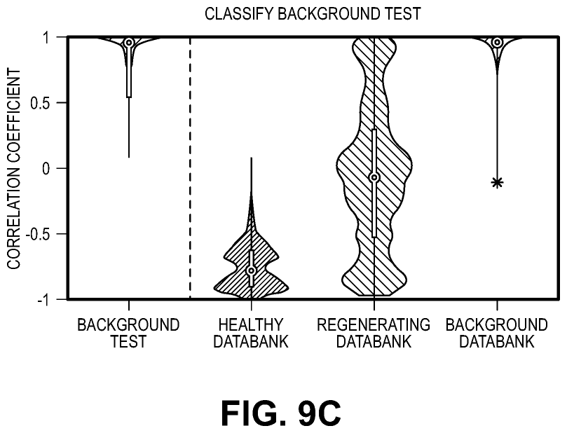

FIGS. 9A-9C illustrate the results of the multispectral nerve classification algorithm, according to an embodiment. FIG. 9A shows a healthy class test set correlated with itself (according to Equation 3) and with reference databanks from three classes: healthy nerve, regenerating nerve, and a plastisol background (according to Equations 1 and 2). The asterisk indicates the reference databank with the closest median to the median of the test set. Therefore, among the reference databanks, the healthy test set is closest to the healthy databank, and the classification of this testing set is healthy (which is the correct class). Similarly, FIG. 9B shows a regenerating class test set correlated with itself (according to Equation 3) and with reference databanks from the same three classes noted above (according to Equations 1-2). The asterisk indicates the reference databank with the closest median to the median of the test set. Therefore, among the reference databanks, the regenerating test set is closest to the regenerating nerve databank, and the classification of this testing set is regenerating nerve (which is the correct class). Likewise, FIG. 9C shows a background class test set correlated with itself (according to Equation 3) and with reference databanks from the same three classes noted above (according to Equations 1-2). The asterisk indicates the reference databank with the closest median to the median of the test set. Therefore, among the reference databanks, the background test set is closest to the plastisol background databank, and the classification of this testing set is plastisol background (which is the correct class).

### Optical Absorption Spectra and Corresponding In Vivo Photoacoustic Visualization of Exposed Peripheral Nerves

Multispectral photoacoustic imaging has the potential to identify lipid-rich, myelinated nerve tissue in an interventional or surgical setting (e.g., to guide intraoperative decisions when exposing a nerve during reconstructive surgery by limiting operations to nerves needing repair, with no impact to healthy or regenerating nerves). Lipids have two optical absorption peaks within the NIR-II and NIR-III windows (i.e., 1000 to 1350 nm and 1550 to 1870 nm wavelength ranges, respectively) that can be exploited to obtain photoacoustic images. However, nerve visualization within the NIR-III window is more desirable due to higher lipid absorption peaks and a corresponding valley in the optical absorption of water. The first known optical absorption characterizations, photoacoustic spectral demonstrations, and histological validations are described below to support in vivo photoacoustic nerve imaging in the NIR-III window.

Four in vivo swine peripheral nerves were excised, and the optical absorption spectra of these fresh ex vivo nerves were characterized at wavelengths spanning 800 to 1880 nm, to provide the first known nerve optical absorbance spectra and to enable photoacoustic amplitude spectra characterization with the most optimal wavelength range. Prior to excision, the latter two of the four nerves were surrounded by aqueous, lipid-free, agarose blocks (i.e., 3% w/v agarose) to enhance acoustic coupling during in vivo multispectral photoacoustic imaging using the optimal NIR-III wavelengths (i.e., 1630 to 1850 nm) identified in the ex vivo studies.

There was a verified characteristic lipid absorption peak at 1725 nm for each ex vivo nerve. Results additionally suggest that the 1630 to 1850 nm wavelength range can successfully visualize and differentiate lipid-rich nerves from surrounding water-containing and lipid-deficient tissues and materials. Photoacoustic imaging using the optimal wavelengths identified and demonstrated for nerves holds promise for detection of myelination in exposed and isolated nerve tissue during a nerve repair surgery, with possible future implications for other surgeries and other optics-based technologies.

There are multiple potential causes of peripheral nerve injury, including trauma, intraoperative procedures, and iatrogenic injury. For example, limb trauma patients can suffer from chronic peripheral nerve injury, the majority of which are caused by motor vehicle accidents. Intraoperatively, nerves can be accidentally injured by mechanisms, such as direct transection (e.g., being mistaken as other tissues), stretch or compression (e.g., a retractor keeping the surgical area exposed, placement of orthopedic implants, improper patient positioning), and thermal damage (e.g., nearby coagulation). In addition, patients receiving nerve blockades can also suffer from nerve damage if the anesthetic needle is not correctly placed relative to the targeted peripheral nerves. Over time, these various groups of patients can suffer from symptoms such as chronic motor dysfunction, sensory dysfunction, decreased dexterity, or pain, and they are eventually referred to specialized centers to diagnose and treat nerve injury, where treatment often includes surgery to expose, isolate, and repair the nerve.

Surgical repair of these injuries remains the mainstay of treatment. However, pre-operative and intra-operative decision-making pertaining to the need, timing, and precise location and nature of surgical intervention is severely limited by the inability to visualize the presence and location of regenerating axons within the affected nerve tissue. Many nerve injuries are closed traction or crush injuries where the affected nerves remains in continuity but the axons within them are disrupted. A subset of patients with these types of injuries will spontaneously recover with time via the slow process of axonal regeneration which begins at the injury site and progresses at a rate of approximately 1 mm per day. Therefore, to avoid unnecessary and potentially harmful surgical intervention, the typical management of these types of injuries begins with a period of 3-6 months of observation to allow for spontaneous recovery to occur. For the patients who ultimately do not recover, this delay in surgical repair downgrades motor functional recovery due to progressive irreversible atrophy of the denervated muscle that takes occurs during the observation period.

To prevent or treat nerve injury, surgeons, anesthesiologists, and other clinicians primarily rely on known anatomical relationships and electrodiagnostics studies. However, known anatomical relationships become distorted when anatomy is disrupted during a surgery or when operating on diseased or damaged tissues. Electrodiagnostic studies include electromyography (EMG) and nerve conduction studies (NCS), which monitor skeletal muscle activity and nerve conduction ability, respectively. These electrodiagnostic studies may be used preoperatively to ascertain whether axons have reached the affected skin and muscle. Combining EMG and NCS with the stimulation of nerve tissue provides an indication of general nerve proximity and general nerve function, but it suffers from imprecise assessments of nerve localization and nerve architecture. In addition, EMG is restricted to identifying only a subset of nerves (i.e., EMG is ineffective at locating sensory nerves, and it is similarly ineffective at locating motor nerves that may not stimulate normally due to prior trauma or intraoperative paralytics). Intraoperatively, direct nerve stimulation may also result in corresponding muscle contraction if the muscle is innervated. Importantly, neither of these diagnostic modalities provide information regarding whether axons are in transit and are regenerating beyond the injury site but have yet to reach the target end organs.

Imaging techniques are increasingly being explored with promising potential to address the above-stated challenges. For example, ultrasound imaging can be used in surgical planning to preoperatively map nerve locations and determine a suitable surgical pathway. In an interventional or intraoperative setting, ultrasound imaging has the potential to offer real-time visualization of nerves and their proximity to needles or other foreign bodies, such as orthopedic implants. However, nerves can be confused with the similar appearance of nearby soft tissue structures in ultrasound images, and changes in local anatomy can alter the expected nerve appearance. In addition, needle tips can be difficult to identify in ultrasound images. The success of ultrasound imaging is also subject to operator skill and expertise. Magnetic resonance imaging (MRI) is an alternative option to provide excellent soft tissue contrast and nearby anatomical landmarks with less dependence on operator performance. However, MRI is expensive, time-consuming to implement, and unsafe to operate in the presence of ferromagnetic metallic devices or implants. Near-infrared fluorescence image-guidance has also been proposed, but it requires the injection of contrast agents. In addition, standard imaging modalities (MRI, ultrasound) provide gross morphologic information but are not able to visualize myelinated axons within nerve tissue. If surgeons were able to intraoperatively determine the location of myelinated axons within the affected nerve(s), early surgical exploration would become warranted and result in substantially improved outcomes for those patients requiring surgical repair. Furthermore, intraoperative decision regarding the precise location and nature of the surgical intervention would be dramatically improved.

Photoacoustic imaging is a promising alternative to existing options for interventional localization of nerve tissue during nerve repair surgeries (as well as during nerve block injections and routine surgeries). To implement photoacoustic imaging, pulsed light is transmitted and selectively absorbed based on the inherent optical properties of endogenous chromophores or metallic needle tips and then converted to acoustic energy, with an ultrasound transducer receiving the resulting signals. In nerve tissue, which contains axons wrapped in insulating myelin sheaths and bundled together by collagenous connective tissue and fatty deposits, the lipids within the myelin sheaths and fat cells preferentially absorb transmitted light to achieve photoacoustic images of nerves.

Photoacoustic imaging of nerves is challenged by the comparable optical absorption properties of lipids and surrounding tissue at singular wavelengths. These similar properties reduce target contrast and impede photoacoustic monitoring of nerves. As a possible solution, lipids are known to have unique optical absorption spectra as a function of multiple illuminating wavelengths, which can be measured by the relative amplitude of the photoacoustic signal at each wavelength (i.e., the photoacoustic amplitude spectra).

Excitation wavelengths that exploit the optical absorption peaks of lipids at 1210 and 1730 nm were previously identified as optimal for photoacoustic imaging of lipid-rich nerve tissue. These wavelengths reside in the second near infrared optical window of approximately 1000 to 1350 nm, hereafter referred to as NIR-II, and in the third near infrared optical window of approximately 1550 to 1870 nm, hereafter referred to as NIR-III. Using these wavelength ranges, the photoacoustic amplitude spectra measured from ex vivo peripheral nerve tissues were compared with the optical absorption of lipid samples, such as subcutaneous fat.

Agreement between photoacoustic amplitude spectra and lipid optical absorption spectra confirmed both the identification of the lipids in nerve tissue and the differentiation of nerve tissue from other structures, such as collagenous tendons and hemoglobin-rich blood vessels. Multispectral photoacoustic imaging may be used to differentiate an in vivo femoral nerve from the femoral artery in a mouse model using a subset of NIR-II wavelengths (i.e., 1100 to 1250 nm). The photoacoustic signal acquired with one NIR-II wavelength (i.e., 1210 nm) may be compared with that acquired with a subset of NIR-III wavelengths (i.e., 1600 to 1850 nm), which demonstrates that the photoacoustic signal from ex vivo intramuscular goat fat was five times greater when targeting the lipid absorption peak at 1730 nm, rather than the lipid absorption peak at 1210 nm. In addition, the 1730 nm lipid absorption peak resides in a valley of the absorption spectra of water, thus providing a biological optical window to target the lipids within nerve tissue.

The success of these previous photoacoustic nerve imaging approaches required two essential components. First, the range of excitation wavelengths was strategically chosen to produce photoacoustic amplitude spectra of nerves that are distinguishable from the photoacoustic amplitude spectra of surrounding tissues. Second, reference lipid optical absorption spectra were available to confirm which measured photoacoustic amplitude spectra originated from nerve tissue. However, no previous work has performed in vivo photoacoustic nerve imaging with wavelengths that leverage the 1730 nm lipid peak (which was identified as preferred over the 1210 nm peak in a previous ex vivo study). In addition, the optical absorption spectra of isolated lipids were previously used to determine that the likely source of the visualized signals was nerve tissue. Characterizing the optical absorption of myelinated nerve tissue may improve both wavelength selection and nerve tissue identification with photoacoustic imaging, particularly when compared with the current information available with the optical absorption spectra of isolated lipids.

The present disclosure presents the first known characterization of the optical absorbance spectra of fresh myelinated nerve samples using a wide spectrum of wavelengths (i.e., 800 to 1880, which span the NIR-I, NIR-II, and NIR-III optical windows). The present disclosure also presents the first known in vivo visualization and corresponding optical absorption characterization of nerve contents using multispectral photoacoustic imaging and the identified optimal wavelengths for composite nerve tissue (i.e., wavelengths 1630 to 1850 nm in the NIR-III optical window). These optimal wavelengths for photoacoustic imaging of composite nerve tissue are hereafter defined as the “NIR-III nerve window.” In addition, the structural and anatomical content of the imaged in vivo nerve tissue was confirmed with histology. The present disclosure focuses on nerves in isolation, rather than nerves surrounded by competing photoabsorbers because it is important to understand the properties of nerve tissue alone, prior to understanding interactions and differentiation from surrounding media. In addition, a single isolated nerve provides an excellent ground truth for in vivo investigations. These fundamental contributions are intended to build the scientific foundation necessary to benefit multiple optics-based nerve visualization and assessment applications.

### Methods

### Nerve Samples

A total of eight peripheral nerve samples were harvested from three Yorkshire swine, hereafter referred to as swine I, swine II, and swine III. These nerve samples consisted of one regenerated median nerve (i.e., 1 year after injury and repair) dissected into two samples from swine I (i.e., two median nerve samples) and one ulnar nerve dissected into two samples each from swine I, swine II, and swine III (i.e., six control ulnar nerve samples). Of the two regenerated nerve samples, one was used for spectroscopic absorbance measurements, and one was used for histological analysis. Of the six control nerve samples, three (i.e., one from each swine) were used for spectroscopic absorbance measurements, and the remaining three were used for histological validation. The samples from swine II and III were available for these measurements prior to those from swine I, which was imaged after the availability of these measurements.

Additional details regarding spectroscopic absorbance measurements, in vivo photoacoustic imaging, and histological validation are described below. The regenerated nerve samples represent nerve anatomy that deviates from normal (e.g., in patients recovering from nervous system diseases or nerve injuries), and the control samples represent normal.

### Spectroscopic Absorbance Measurements

Absorbance measurements were obtained with a Cary 5000 dual beam spectrophotometer and external diffuse reflectance accessory (DRA) from Agilent Technologies (DRA-2500, Santa Clara, California) for the following five samples: (1) powdered cholesterol (C8667, Sigma-Aldrich, St. Louis, Missouri); (2) 3% w/v agarose (A6013, Sigma-Aldrich, St. Louis, Missouri), (3) phosphate buffered saline (PBS), which is a water-based salt solution (10010023, Thermo Fischer Scientific, Waltham, Massachusetts); (4) three fresh swine ulnar nerves; and (5) one fresh swine median nerve. Fresh is defined as spectroscopic absorbance measurements being performed≤24 h after harvesting. For example, samples were mounted for placement in a DRA centermount port. The cholesterol sample (representing the primary lipid in the myelin sheath of nerves) was deposited between two glass slides. This cholesterol sample was included to provide a baseline reference point for nerve measurements using the same equipment and process. To mount the nerves for spectroscopic analysis, a 3D-printed sample holder was designed with a rectangular cavity to hold the nerve in place during data acquisition. PBS was added to the rectangular cavity to minimize air interfaces, and coverslips were attached to each side of the nerve sample holder. The coverslips secured the nerve inside the holder and limited dehydration of the tissue.

To obtain the PBS absorbance measurement (which represents the absorbance of water), the rectangular cavity of the sample holder was filled with PBS and secured with two coverslips. To obtain the agarose absorbance measurements, the agarose sample was sliced from an agarose block and adhered to a coverslip on a rectangular slit sample holder. Prior to sample measurements, 100% and 0% absorbance baselines were obtained. For each sample, the 100% absorbance baseline was obtained by completely blocking the beam path into the DRA. The 0% absorbance baselines for each sample were obtained using an appropriate “blank” (i.e., two bare glass slides for cholesterol and an empty rectangular slit sample holder for PBS, agarose, and nerve). Each sample was placed in the DRA centermount at an incident angle of 5 deg. The wavelength measurement range was 800 to 1800 nm in 1 nm increments (which spanned the NIR-I, NIR-II, and NIR-III optical windows), and the averaging time was 0.1 s per wavelength.

### In Vivo Photoacoustic Imaging

The photoacoustic imaging system consisted of a Phocus Mobile system (Opotek, Carlsbad, California), an E-CUBE 12R ultrasound scanner (Alpinion Medical Systems, Seoul, South Korea), and an Alpinion L8-17 ultrasound transducer. In the Phocus Mobile system, a Q-switched Nd:YAG laser, operating at 10 Hz with a 5 ns pulse width, was coupled to an optical parametric oscillator (OPO). The OPO was operated at idler wavelengths in the NIR-III nerve window, specifically 1630 to 1850 nm in 5 nm increments. This wavelength range was chosen based on the lipid absorption peak reported in the existing literature and the spectroscopic absorbance measurements obtained from nerves of swine II and III. A motorized variable attenuator (MVA) was placed in the beam path between the OPO output and an optical fiber bundle, with 3.9 mm diameter bifurcated ends (Armadillo SIA, Sunnyvale, California). The MVA was calibrated to ensure that the bifurcated ends of the optical fiber bundle emitted a mean total energy of 4.2 mJ/pulse at each wavelength in the NIR-III nerve window (mean fluence of approximately 18 mJ/cm2).

To determine the possible agreement between the amplitude of photoacoustic signals achieved in an in vivo setting and the spectroscopic absorbance measurements for nerve tissue, the ulnar (control) and median (regenerated) nerve samples from swine I were imaged with the photoacoustic imaging system prior to resection. The regenerated median nerve model involved sharp transection of the median nerve with immediate tension-free microsurgical repair using 8-0 nylon epineurial sutures followed by a recovery period of one year. To perform imaging, the ulnar and median nerves in the proximal forelimb were first exposed, and then each underwent epineurectomy to remove the influence of excess connective tissue on the photoacoustic measurements of the nerve tissue.

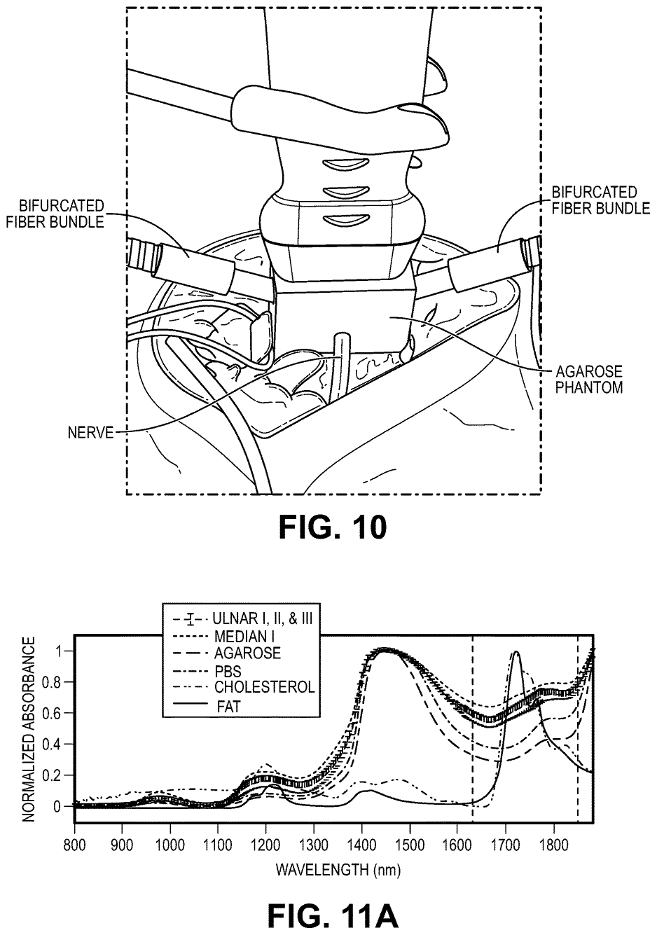

To provide acoustic coupling for photoacoustic imaging, a custom 3% w/v agarose block was placed to surround each nerve, and the bifurcated optical fiber bundle described above was inserted into cavities within the agarose block, as shown in FIG. 10. More particularly, FIG. 10 illustrates an experimental setup for in vivo photoacoustic imaging, including bifurcated fiber bundles secured in a custom-molded agarose block to bilaterally illuminate the nerve, with an ultrasound transducer positioned to image a circular cross section of the illuminated nerve.

In addition to providing acoustic coupling, the custom agarose block also secured the relative positions of the nerve and imaging components for the duration of the experiment and positioned the nerve at a reasonable distance of 1.75 cm from the surface of the ultrasound transducer. The ultrasound transducer was placed orthogonal to the surface containing the opposing fiber bundle cavities within the agarose block to image a circular cross section of the nerve. The imaged nerves were then resected after euthanasia to create the four samples from swine I. These procedures were approved by the Johns Hopkins University Animal Care and Use Committee (Protocol SW20M73).

Co-registered ultrasound and photoacoustic images were generated from channel data with delay-and-sum (DAS) beamforming. To reduce the impact of pulse-to-pulse energy variations, 10 beamformed images per wavelength were averaged to form a single normalized photoacoustic image for spectral analysis at each wavelength. To create nerve and background regions of interest (ROIs) for the spectral analysis, the normalized photoacoustic images acquired with 1230 and 1725 nm wavelengths were first thresholded to display signal amplitudes≥0.15.

Artifacts (i.e., signals surviving the threshold yet located outside of the nerve or agarose-fiber interface locations) were manually removed, the location of remaining signals were combined using the Boolean algebra logical OR operation, and the combined result was separated into nerve and background ROIs. The amplitude of each DAS image pixel within these ROIs was measured as a function of wavelength to generate photoacoustic amplitude spectra for the NIR-III nerve window.

To demonstrate the advantage of the NIR-III nerve window relative to NIR-II wavelengths, the photoacoustic system was additionally operated at the NIR-II wavelengths that are accessible with the system (i.e., 1230 to 1450 nm). Otherwise, the same system, in vivo setup, nerve locations on swine I, and ROIs described above were employed to report the amplitude of each DAS image pixel within the ROIs as a function of NIR-II wavelengths.

### Histological Validation

Toluidine blue staining of the resected nerve samples from swine I, II, and III was performed to provide ground truth anatomical and structural information of the nerve samples, including (1) confirming the presence of lipids in the nerve samples, (2) assessing the lipid distribution within the nerve samples, and (3) quantifying structural differences between nerve samples. In particular, the nerve samples were fixed for 48 h at 4 °C in a solution of 2% glutaraldehyde, 3% paraformaldehyde, and 0.1 M PBS (pH 7.2). Samples were then post-fixed in 2% osmium tetroxide, dehydrated in ascending alcohol series, and embedded in resin prior to staining with 1% Toluidine blue. Representative slices were evaluated using a Zeiss Axioplan 2 microscope (Carl Zeiss Microscopy LLC, White Plains, New York), and images were captured using a Jenoptik ProgRes C5 camera (Jupiter, Florida) mounted to the microscope. At 100× magnification, a single nerve slice produced 4 to 14 non-overlapping micrograph segments. Micrograph segments (.tif files, 2580×1944 pixels, 15 MB) were imported into ImageJ (FIJI Package, version 2.0, NIH, Bethesda, Maryland) and analyzed by an assessor who was blinded to our study goals. Histomorphometric assessment of nerve and myelin anatomy was performed by measuring the nerve density, fiber diameter, myelin thickness, and ratio of the axon diameter to the fiber diameter (i.e., G-ratio). The G-ratio indicates the degree of axon myelination, with a value of 1.0 indicating a completely unmyelinated axon.

### Results

Optical Absorption Characterization with Ex Vivo Samples

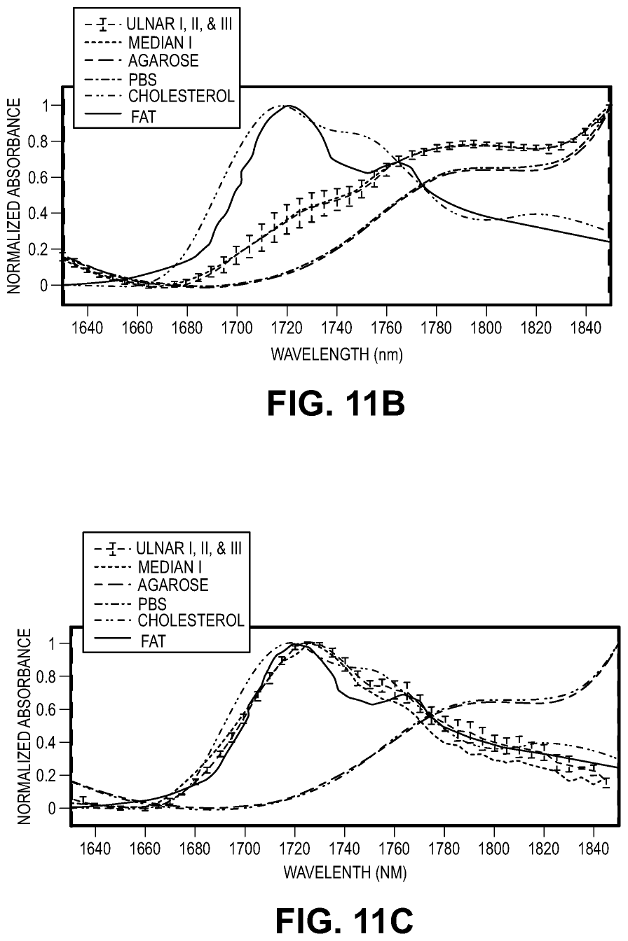

FIGS. 11A-11C show spectroscopic optical absorbance measurements of the five samples noted above (i.e., PBS, 3% w/v agarose, cholesterol, one median nerve, and the average ═one standard deviation of three ulnar nerve samples). More particularly, FIGS. 11A-11C illustrate graphs showing absorbance measurements of three ulnar nerve samples (mean±one standard deviation), one median nerve sample, and samples of powdered cholesterol, 3% w/v agarose, and PBS, shown for (a) 800 to 1880 nm wavelengths, (b) 1630 to 1850 nm wavelengths [indicated by vertical dashed lines in panels (a)-(c)], and (c) 1630-1850 nm wavelengths after the PBS absorbance spectrum was subtracted from each nerve measurement. In each case, the normalized optical absorption of fat was extracted and is shown for comparison.

The absorbance measurements for a wide range of wavelengths measured by the spectrophotometer (i.e., 800 to 1800 nm) are available in FIG. 11A. In the NIR-II range, cholesterol and nerve tissue each have an absorbance peak at 1210 nm, which is similar to that observed in fat and other isolated lipids. Agarose, PBS, and nerve tissue each have an absorbance peak at 1450 nm, which is similar to that of water. In the NIR-III range, cholesterol has a second absorbance peak at 1715 nm, and fat has absorbance peaks at 1720 and 1765 nm, respectively.

To better view the absorbance characteristics of the samples in the NIR-III nerve window [indicated by the dashed lines in FIG. 11A], FIG. 11B presents a zoomed and normalized version of FIG. 11A. Although the agarose, PBS, and nerve samples have similar absorbance measurements in this window of wavelengths (i.e., 1680 to 1850 nm), the nerve absorbance measurements also deviate from that of the water-based samples (i.e., agarose and PBS) and do not contain an expected lipid absorbance peak at 1715 nm, as demonstrated in the cholesterol and fat measurements. Instead, nerve tissue and water-based agarose and PBS absorbance measurements each monotonically increase in the range of 1680 to 1790 nm and reach a plateau in the wavelength range 1790 to 1830 nm, suggesting that water is the dominating chromophore in nerve tissue.

To remove the dominating contribution of water from the nerve absorbance measurements, FIG. 11C shows the nerve absorbance spectra after the PBS absorbance spectrum was subtracted from each nerve measurement. To perform the subtraction, each measured absorbance spectra was first normalized by its maximum value and then scaled to an amplitude range of 0 to 1, and the normalized and scaled PBS absorbance spectrum was subtracted from each normalized and scaled nerve absorbance spectrum. This difference was scaled to an amplitude range of 0 to 1 to create the isolated nerve absorbance spectra shown in FIG. 11C. These isolated nerve absorbance spectra have an absorbance peak at 1725 nm, which differs from the absorbance peak of fat and cholesterol by 5 and 10 nm, respectively. The isolated nerve absorbance spectra also have either a plateau or a decreased rate of change in the range of 1745 to 1765 nm, which is similar to that of fat and cholesterol in the same wavelength range. These similarities and differences highlight expected subtleties between the absorption spectra of various lipid-rich structures. The presence of the observed 1725 nm peak and surrounding absorption profile for composite nerve tissue also informs optimal wavelengths to employ when acquiring photoacoustic images of nerves. In addition, the results in FIGS. 11A-11C demonstrate that both water and lipid contribute to the absorbance spectra of the ulnar and median nerve samples.

In Vivo Photoacoustic Characterizations and Comparisons with Ex Vivo Spectrophotometer Results

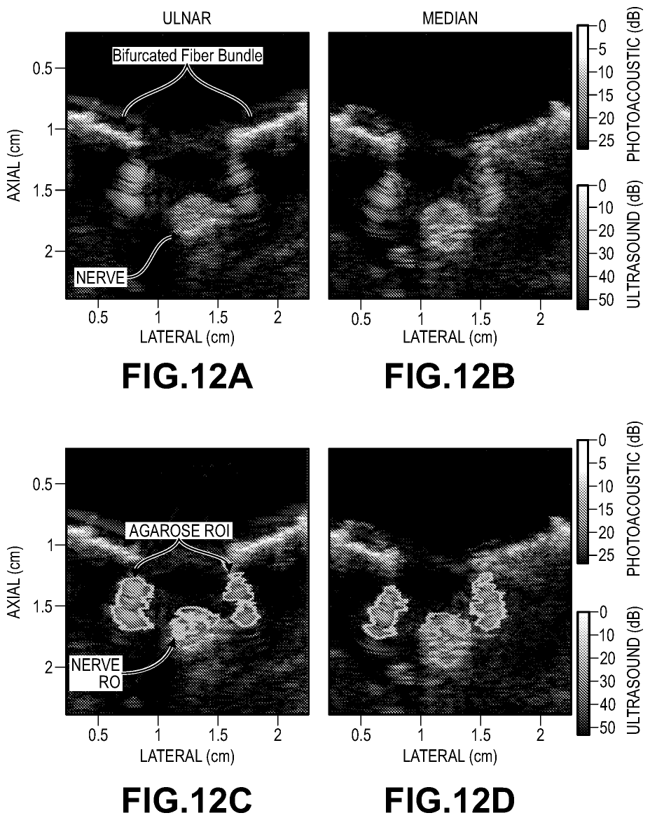

FIGS. 12A-12D show co-registered photoacoustic and ultrasound images of the ulnar and median nerves from swine I when bilaterally illuminated with an optical wavelength of 1725 nm. Photoacoustic signals are primarily present in the superficial, superior area of the circular nerve cross section in FIGS. 12A and 12B. In addition, photoacoustic signals are observed immediately distal to the bifurcated light sources, which indicate photoacoustic signals originating from the agarose block. FIGS. 12C and 12D highlight the ROIs utilized to measure the photoacoustic amplitude spectra, with details on ROI selection.

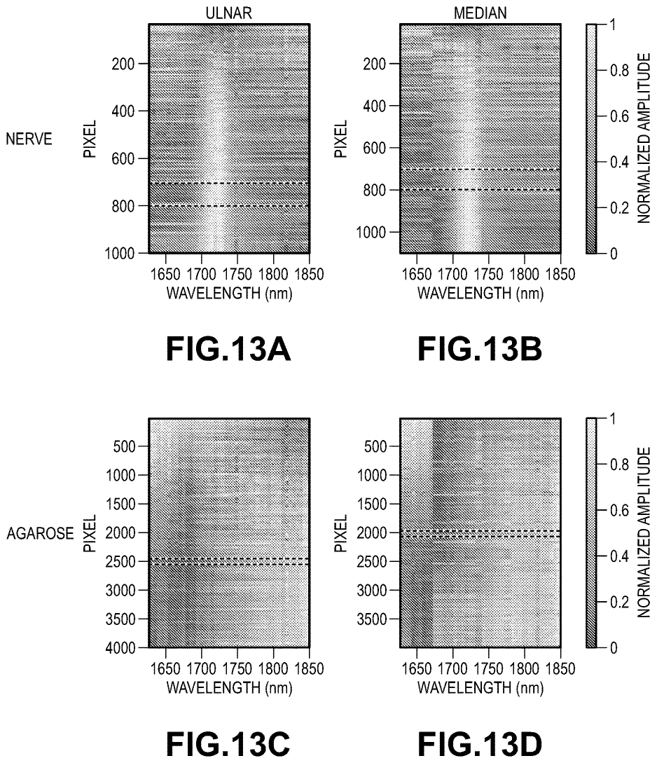

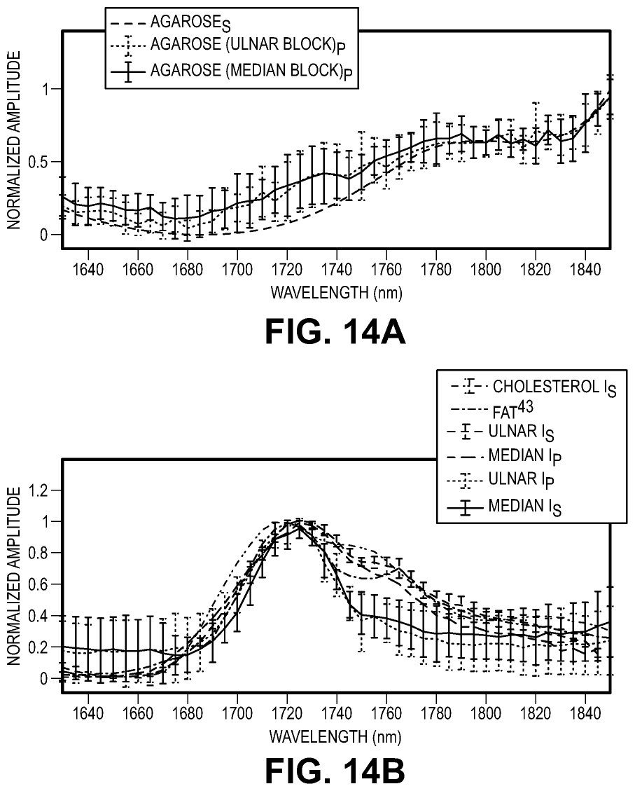

FIGS. 13A-13D show the measured photoacoustic amplitude spectra of each pixel within the ROIs shown in FIGS. 12C and 12D. More particularly, FIGS. 13A-13D illustrate in vivo photoacoustic amplitude spectra of the samples computed for each pixel within the ROI for (13A) ulnar nerve I, (13B) median nerve I, (13C) the agarose block used to image ulnar nerve I, and (13D) the agarose block used to image median nerve I. The photoacoustic amplitude spectrum of each pixel is normalized between 0 and 1. Dashed white lines outline data extracted to create FIGS. 14A and 14B.

The photoacoustic amplitude spectra of the ulnar and median nerve each have a peak at 1725 nm. In direct contrast, the photoacoustic amplitude spectra of the corresponding agarose blocks do not have a single characteristic peak in the NIR-III nerve window. Instead, the photoacoustic amplitude spectra of both agarose blocks show increased photoacoustic amplitude at the lower and upper boundaries of the NIR-III nerve window (i.e., 1630 to 1675 nm and 1740 to 1850 nm, respectively) with a valley in the 1675 to 1740 nm range.

The dashed lines in FIGS. 13A-13D (i.e., independent groupings of 100 pixels that maximized the dominant peak signal location and minimized the standard deviation outside of each dominant peak signal of interest) indicate regions utilized to plot photoacoustic amplitude spectra for direct comparison with spectrophotometer measurements.

FIGS. 14A and 14B directly compare the mean #one standard deviation of the photoacoustic amplitude spectra residing within the horizontal dashed lines in FIG. 13A-13D and the corresponding absorbance spectra replicated from FIGS. 11A-11C. There is general agreement between the absorbance spectra and photoacoustic amplitude spectra of agarose in FIG. 14A. In addition, the photoacoustic amplitude spectra of agarose are reproducible across the two manufactured agarose blocks. FIG. 14B compares the photoacoustic amplitude spectra of the ulnar and median nerves with the absorbance measurements of the ulnar and median nerve samples after the PBS absorbance spectra were subtracted from each nerve measurement. The photoacoustic amplitude spectra of the ulnar and median nerves have a peak at 1725 nm, which agrees with the 1725 nm peak in the corresponding absorbance spectra. This peak differs from that of fat and cholesterol by 5 and 10 nm, respectively.

Comparisons of Photoacoustic Amplitude Spectra within NIR-II and NIR-III Wavelengths

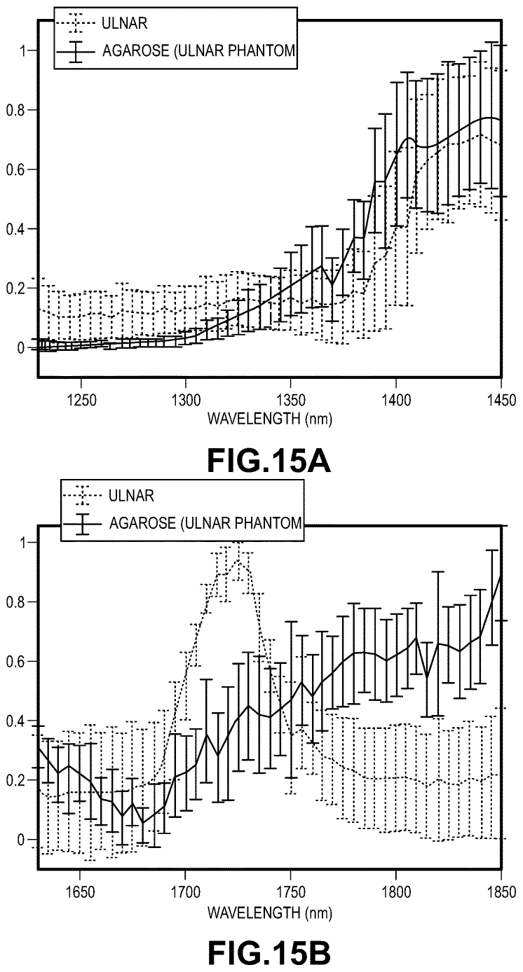

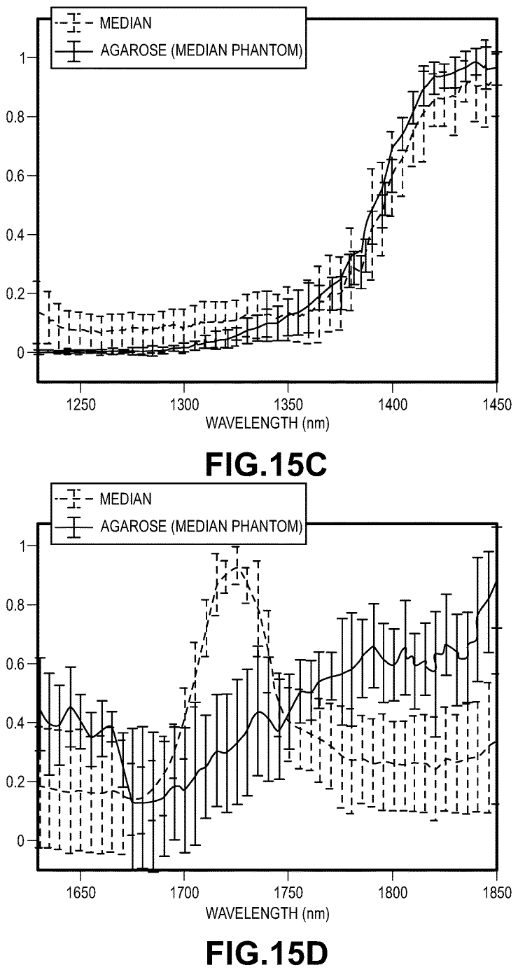

FIGS. 15A-15D show a comparison of the NIR-II and NIR-III photoacoustic amplitude spectra for the in vivo median and ulnar nerves from swine I and surrounding agarose. More particularly, FIGS. 15A-15D show a comparison of photoacoustic amplitude spectra at NIR-II (1230 to 1450 nm) and NIR-III (1630 to 1850 nm) wavelengths, acquired from swine I. (15A), (15B) Ulnar nerve compared with agarose surrounding the ulnar nerve. (15C), (15C) Median nerve compared with agarose surrounding the median nerve. NIR-III results are duplicated from FIGS. 14A and 14B (i.e., mean±one standard deviation of the photoacoustic amplitude spectra) with agaorose and nerve results overlapped on the same plots.

The results for the NIRIII wavelengths [FIGS. 15A and 15D] are the same as that shown in FIGS. 14A and 14B with agaorose and nerve results overlapped on the same plots. At the lower NIR-II wavelengths [FIGS. 15A and 15C], the nerve has similar optical properties to the mostly water-based agarose material, due to more dominance from the water absorption spectrum. As a result, distinguishing water from nerves at these NIR-II wavelengths is anticipated to be difficult. However, at the higher wavelengths, differentiation from water was possible due to the corresponding valley in the absorption spectrum of water, which can be appreciated from the agarose and PBS spectra in FIGS. 11A-11C.

### Ex Vivo Histological Assessments

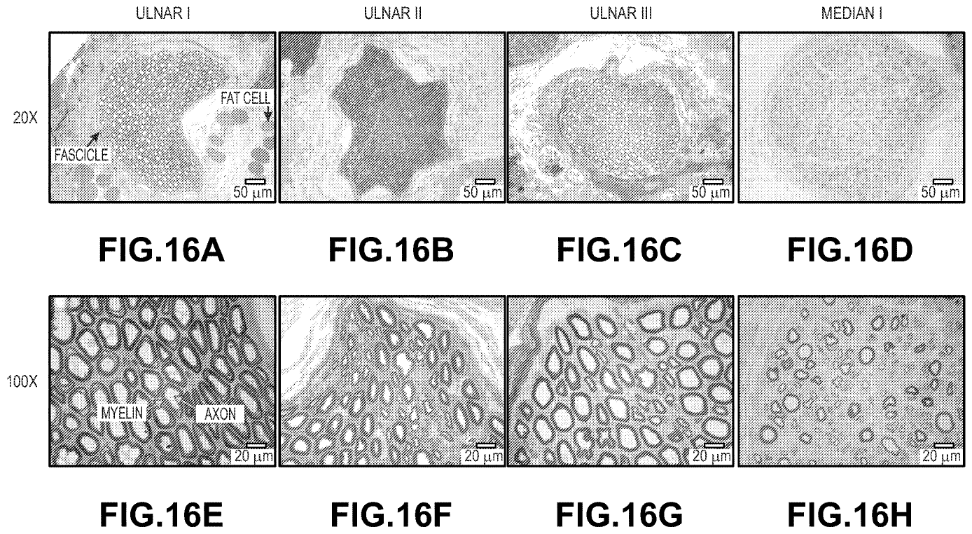

FIGS. 16A-16H show representative histological sections of ulnar and median nerve samples from swine I. More particularly, FIGS. 16A-16H show histology sections of the ulnar nerves from (16A), (16E) swine I; (16B), (16F) swine II; (16c), (16G) swine III; and (16D), (16H) the median nerve from swine I, stained with toluidine blue to determine the presence of myelin, and visualized with (a)-(d) 20× and (e)-(h) 100× magnification.

In the histological sections visualized with 20× magnification, the toluidine blue staining indicates that myelin is contained within the fascicles for each nerve sample. The staining also outlines the shape of the fascicles and demonstrates differences in fascicle shape between the nerve samples, from circular [e.g., FIGS. 16A and 16D] to irregularly shaped [e.g., FIGS. 16B and 16C]. In addition, fat cells are observed near the myelin-filled fascicles. The presence of fat cells indicates that both the lipid content of the fat cells and the lipid content of the myelin sheaths are the most likely contributors to the optical absorption characteristics of composite nerve tissue. In the histological sections of FIGS. 16A-16H visualized with 100× magnification, the toluidine blue staining demonstrates the anatomy of the myelin sheath in which it encases each axon in each nerve sample. Qualitatively, axon diameter, myelin thickness, and fiber density were similar among the ulnar nerve samples. However, the regenerated median nerve has reduced axon diameter, myelin thickness, and fiber density when compared with these same properties in the ulnar nerve section.

TABLE 1
Histomorphometrics (average ± one standard
deviation) of ex vivo ulnar and median nerve samples.

| | Ulnar I | Ulnar II | Ulnar III | Median I |
|---|---|---|---|---|
| Nerve density (fibers/mm²) | 4688 ± 2755 | 8250 ± 2353 | 6944 ± 2837 | 8661 ± 2961 |
| Fiber diameter (μm) | 14.41 ± 4.41 | 12.33 ± 2.26 | 13.48 ± 3.44 | 9.44 ± 2.36 |
| Myelin thickness (μm) | 5.63 ± 1.04 | 5.21 ± 1.50 | 5.53 ± 0.94 | 3.68 ± 0.88 |
| G-ratio | 0.59 ± 0.105 | 0.58 ± 0.095 | 0.58 ± 0.094 | 0.60 ± 0.067 |

Table 1 quantitatively compares the average nerve density, fiber diameter, myelin thickness, and G-ratio measured for all micrograph segments of each nerve sample. In particular, the average nerve density of the regenerated median nerve is greater than that of each ulnar nerve, whereas the average fiber diameter and average myelin thickness of the regenerated median nerve are lower than that of each ulnar nerve. The average G-ratio demonstrates a similar degree of myelination among the ulnar and median nerve samples.

### DISCUSSION

This work is the first to characterize the optical absorbance spectra of fresh swine nerve samples using a wide spectrum of wavelengths (i.e., 800 to 1880). In addition, in vivo visualization of healthy and regenerated swine nerves with multispectral photoacoustic imaging was demonstrated for the first time in the NIR-III nerve window. The absorbance of nerve tissue was unexpectedly dominated by the presence of water, as indicated by FIGS. 11A and 11B. However, when a possible solution to address this observation was implemented by subtracting the normalized water contribution from the normalized spectrophotometer measurements (which does not depend on known concentration ratios), a characteristic lipid peak at 1725 nm was observed [FIG. 11C].

This 1725 nm peak is unique to nerve tissue as other lipids including fat and cholesterol had lower absorption peaks at 1720 and 1715 nm, respectively [FIG. 11C]. Similar observations of differences among the spectroscopic absorbance measurements of different lipids were previously observed between beef fat and sunflower oil and between perivascular fat and olive oil. It was initially hypothesized that characterization of the absorbance of composite nerve tissue is advantageous for identification of nerve tissue, particularly when compared with the absorbance of other isolated lipids. This hypothesis is confirmed based on the agreement between the 1725 nm peak in the spectroscopic absorbance measurements and the 1725 nm peak in the in vivo photoacoustic amplitude spectra (e.g., FIGS. 14A-14D). In addition, the 5 to 10 nm differences in the location of this peak in comparison with other lipids (e.g., cholesterol, murine liver fat, intramuscular goat fat) further support our hypothesis.

The comparison of the photoacoustic amplitude spectra of agarose in FIGS. 14A and 14B and nerve in FIG. 14B reveals an overlap of spectral amplitudes at 1655 and 1750 nm wavelengths. This overlap is more clearly demonstrated in FIGS. 15B and 15D, which show the same data in a single plot for each nerve. However, the observation of the photoacoustic amplitude spectra across the entire NIR-III nerve window demonstrates the unique spectral features of nerve and agarose, including the 1725 nm absorption peak of nerve tissue. The presence or absence of these and other spectral features in multispectral photoacoustic measurements can be exploited to identify the photoacoustic signal as originating from nerve, rather than other surrounding tissue or materials in an in vivo setting (e.g., when implementing novel and conventional spectral unmixing approaches). These advantages of the NIR-III window are less likely to be beneficial if trying to differentiate nerve from water at the lower NIR-II wavelengths, as demonstrated in FIGS. 15A and 15C.

Specific clinical applications that have the potential to benefit from the ability to identify the presence of a nerve using the 1725 nm spectral peak observed in vivo and confirmed with ex vivo spectroscopic measurements include iatrogenic nerve injury prevention, extraneural needle localization, and nerve repair surgery. In particular, nerve tissue has been differentiated from blood and tendons, albeit with lower wavelengths (e.g., 690 to 1260 nm and 1160 to 1260 nm, respectively). The combination of this with the new findings presented herein supports the introduction of future clinical solutions that multiplex between lower wavelengths and the higher 1725 nm wavelength (e.g., to prevent iatrogenic nerve injury when multiple tissues are present). One may also use a combination of wavelengths that includes 1725 nm to confirm an extraneural (rather than intraneural) needle location for peripheral nerve block injections to avoid permanent nerve damage from the injection, which can be achieved with an optical fiber located inside the injection needle. With this setup, location information about the needle tip and distinction between different tissues and chromophores are anticipated to be possible with a combination of ultrasound and photoacoustic imaging. Robotic approaches may additionally be introduced to minimize operator dependence. A third possible clinical implementation is to expose a nerve during plastic or reconstructive surgery (e.g., during nerve repair) to ensure that a user (e.g., surgeon) only operates on nerves needing repair, rather than healthy or regenerating nerves. Depending on the clinical application and surrounding environment, lower wavelengths can be used when nerves are surrounded by other tissues, whereas higher wavelengths have the potential to offer greater photoacoustic amplitude-related feature detection when nerves are isolated [see FIG. 11A]. With new results that provide optical absorbance characterization, wavelength selection, and corresponding demonstrations of the feasibility of in vivo photoacoustic imaging of isolated nerves, our contributions successfully establish a new scientific foundation to be leveraged by future clinical applications.

Histology confirmed that the likely source of the 1725 nm absorption peak was the presence of lipids within the myelin sheath and fat cells of nerve tissue (FIGS. 16A-16H). Histomorphmetrics reveal overlapping mean±standard deviation nerve fiber density and G-ratio (i.e., degree of myelination) between the regenerated median nerve samples and the control ulnar nerve samples (Table 1). Comparisons across spectroscopic absorbance measurements and photoacoustic amplitude spectra similarly revealed negligible optical absorption changes between the control (i.e., ulnar) and regenerated (i.e., median) nerve samples. Given this similarity, our newly introduced nerve optical absorption characterization results may be employed as a future reference spectrum for photoacoustic imaging of other similarly myelinated nerves (i.e., other than healthy ulnar and regenerated median nerves) and may also be utilized for other optics-based nerve imaging techniques (e.g., diffuse reflectance spectroscopy). As with any optics-based application, there are also possible cases in which the information provided by the 1725 nm peak may not be useful (e.g., if there is significant spectral overlap with other chromophores of interest, as observed in FIGS. 15A-15D when using NIR-II wavelengths).

As noted above, purely optical imaging methods have the potential to utilize the new optical characterizations presented herein. The benefits of photoacoustic imaging relative to purely optical imaging methods include better penetration depth and spatial resolution. Although the optical penetration depth in FIGS. 12A-12D appears to be approximately 1 to 2 mm from the surface of the nerve when illuminated with a wavelength of 1725 nm and a mean fluence of approximately 18 mJ/cm², the ANSI limit for a wavelength of 1725 nm is 1 J/cm² when imaging through skin. Therefore, higher energies are likely to achieve greater penetration depths than that presented in FIGS. 12A-12D.

Despite the agreement between the optical absorption peak of nerves at 1725 nm in the spectroscopic absorbance measurements and photoacoustic amplitude spectra [FIG. 14B)], two discrepancies were observed between the datasets. First, although absorbance measurements of nerve tissue were dominated by water absorption [FIG. 11B], photoacoustic amplitude spectra measurements of nerve tissue were dominated by lipid absorption [FIG. 14B]. A possible reason for this discrepancy is the presence of excess PBS on and surrounding the nerve in the sample holder due to the nerve being transported from the operating room to the spectroscopy machine in a container filled with PBS. Second, the amplitude of the spectroscopic absorbance measurements of nerve tissue in the range of 1745 to 1765 nm is greater than that of the corresponding normalized photoacoustic amplitude spectra [FIG. 14B].

Temperature differences between the ex vivo and in vivo nerve samples are one potential cause of this difference, particularly when considering that the optical absorption of chromophores such as water can vary with temperature. Nonetheless, the observed differences do not affect the major conclusions. Users of the proposed technology are likely to be most interested in peak values, which are consistent across both spectrophotometer and photoacoustic measurements (and are also consistent with the spectrophotometer results obtained from a myelinated phrenic nerve sample).

Future work will be dedicated to additional technology development, such as the implementation of improved light delivery designs and photoacoustic-based diagnosis of nerve health (e.g., degree of myelination). In particular, the experimental procedures implemented to isolate in vivo nerves are similar to the surgical approach used to expose and treat nerve injuries. Therefore, the proposed approach is promising for the development of new technology catered to nerve imaging during nerve repair surgeries within the NIR-III nerve window that has now been characterized for the first time, to the authors' knowledge.

### CONCLUSION

The work presented in this disclosure provides foundational evidence to support an optimized in vivo multispectral photoacoustic nerve imaging approach using NIR-III wavelengths. Previously, this type of information was available for ex vivo samples or for lipids that we now know do not share the exact same spectral profile as composite nerve tissue. Spectroscopic and photoacoustic imaging analyses demonstrated that exploiting the characteristic 1725 nm absorption peak of nerve tissue enables the identification of nerve tissue and the differentiation of nerve tissue from other substances, such as aqueous materials (e.g., our custom agarose block). Although histology identified structural differences between the regenerated and control nerve samples (e.g., fiber diameter, myelin thickness), the combination of spectroscopic, photoacoustic imaging, and histomorphmetric G-ratio (i.e., degree of myelination) analyses revealed negligible differences between the regenerated and control nerve samples due to the similar presence of lipids within each myelinated nerve. These results highlight the clinical promise of multispectral photoacoustic imaging as an intraoperative technique to determine the presence of, and prevent iatrogenic injury to, myelinated nerves, with possible future implications for other optics-based technologies.

### SUPPLEMENTARY MATERIAL

### Normalized Subtraction of Spectrophotometer Measurements

To account for the unexpected contribution of PBS in FIGS. 11A-11C, each absorbance spectra was normalized and scaled from 0 to 1, then the normalized and scaled PBS absorbance spectrum was subtracted from each normalized and scaled nerve absorbance spectra measurement. The final result was then rescaled for display in FIG. 11C.

The rationale supporting this implementation relies on three assumptions to recover local optical absorption peaks associated with a single chromophore component in a mixture of chromophore components:
1. One component is more similar to the mixture than the mixture is to the component(s) being recovered.
2. The peak of the absorption spectrum of one component occurs in the valley of the absorption spectrum of other components.
3. The peaks of the absorption spectra of the components in the mixture occur at different wavelengths.

The first assumption is true for the nerve results because the spectrum of water is generally an order of magnitude greater than that of fat, particularly when comparing peak optical absorption values. Therefore, the PBS spectrum in FIG. 11A is more similar to the spectra obtained with nerves surrounded by PBS, when compared to the similarity between the combined spectra (i.e., nerve and PBS) and the lipid spectra in FIG. 11A. The second assumption is true for PBS relative to cholesterol (i.e., the primary lipid in the myelin sheath of nerves) in FIGS. 11A-11C. The third assumption is true for the PBS and lipid spectra within the NIR-III nerve window in FIGS. 11A-11C.

Because these three assumptions are true, yet the ratio of PBS-to-lipid is unknown, the absorbance was normalized to decrease the effects of the ratio of PBS-to-lipid. This ratio may otherwise impact the absorbance (e.g., a higher concentration ratio will yield a higher absorbance across the spectrum), but the shape of the spectral peak is expected to remain the same (e.g., when absorbance is calculated for different concentration ratios, the normalized results are expected to be similar). Therefore, by normalizing and scaling, the effects of concentration may be minimized, and, in this case, knowledge of the PBS-to-lipid ratio is not required to implement the subtraction method described above.

To mathematically describe the logic and implement in silico validation experiments, the known absorption profile of water is μ_{water}, and the known absorption profile of fat is μ_{fat}, with various combinations of water and fat concentrations (i.e., factors of α₁ and α₂, respectively) mathematically described as a linear combination:

C(α₁,α₂)=α₁μ_{water}+α₂μ_{fat} (1)

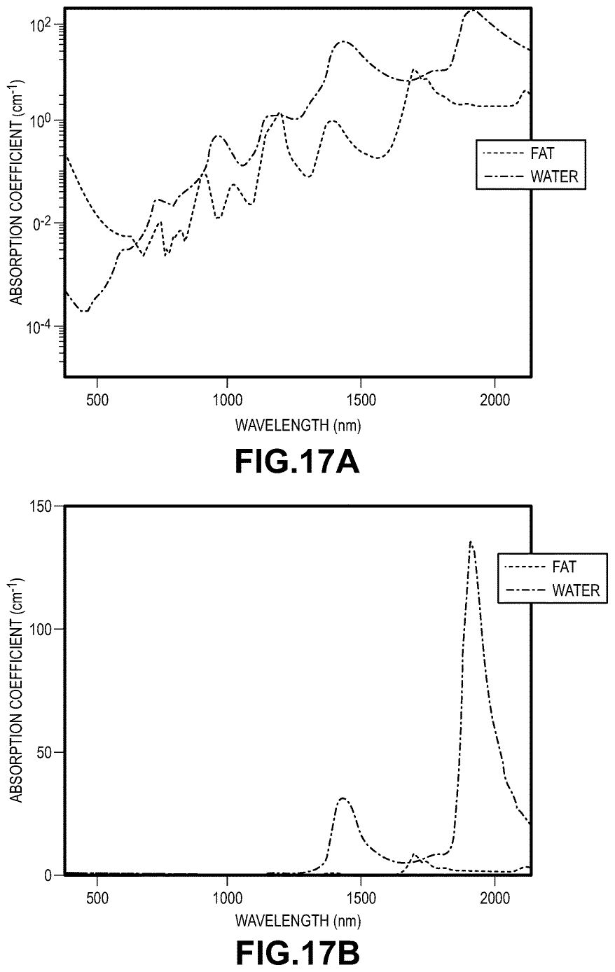

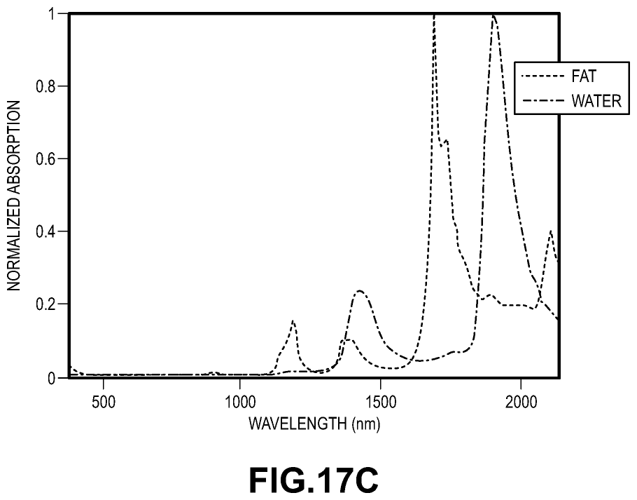

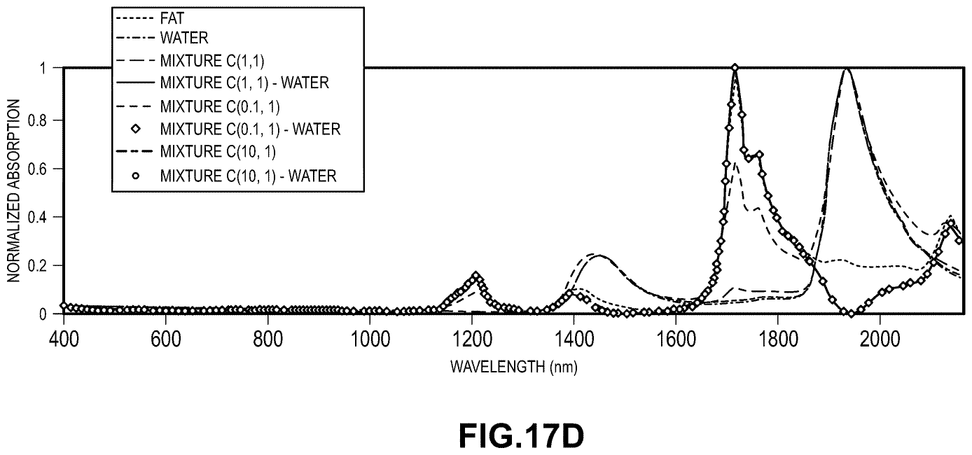

When α₁ is varied to equal 0.1, 1, or 10, while α₂=1, and water is normalized from 0 to 1 then subtracted from the normalized values of C (α₁, α₂), the results in FIGS. 17A-17D are achieved, which each produce a peak that consistently matches the primary of peak of μ_{fat} at 1715 nm wavelength. More particularly, validation of subtracting normalized and scaled PBS absorbance from normalized and scaled nerve absorbance spectra using in silico experiments. The absorption spectra of fat (i.e., a lipid) and water were extracted from and are shown on a (FIG. 17A) log scale, (FIG. 17B) linear scale, and (FIG. 17C) normalized scale. In FIG. 17D, the normalized and scaled absorbance of fat and water are shown with normalized and scaled concentration mixtures described by α₁=0.1, 1, or 10 and α₂=1, based on Eq. (1). The normalized and scaled water spectrum was subtracted from the normalized and scaled mixture spectra, yielding similar results to fat.

There are similarly matching peaks at 1205 and 2135 nm wavelengths in FIG. 17D. Otherwise, the dissimilarities between the resultant subtracted spectrum and the lipid spectrum at wavelengths spanning 1400-1630 nm and 1885-2090 nm occur because there is no local peak to recover (at 1885-2090 nm wavelengths) or the second assumption described above was not met (at 1400-1630 nm wavelengths).

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

1. A system for assessing a nerve in a patient, the system comprising:
an encasement configured to be positioned at least partially around the nerve;
one or more fibers coupled to the encasement, wherein light is transmitted through the one or more fibers, through the encasement, and onto the nerve, and wherein a sound is generated in response to the nerve absorbing the light; and
an ultrasound transducer coupled to the encasement, the one or more fibers, or both, wherein the ultrasound transducer is configured to measure the sound.

2. The system of claim 1, wherein the encasement comprises first and second encasement portions that are configured to actuate between an open position and a closed position, wherein the first and second encasement portions in the closed position are configured to secure the nerve at least partially therebetween.

3. The system of claim 2, wherein the first encasement portion comprises an inner surface, and wherein the inner surface defines a recess in which the nerve is positioned when the first and second encasement portions are in the closed position.

4. The system of claim 3, wherein the inner surface further defines an opening through which the light passes to the nerve.

5. The system of claim 4, wherein the opening extends through the recess.

6. The system of claim 4, wherein a central longitudinal axis through the opening is substantially perpendicular to a central longitudinal axis through the recess.

7. The system of claim 1, wherein the one or more fibers comprise:
a first flexible, bifurcated fiber bundle that transmits a first portion of the light that shines on a first circumferential portion of the nerve; and
a second flexible, bifurcated fiber bundle that transmits a second portion of the light that shines on a second circumferential portion of the nerve, wherein the first and second circumferential portions are circumferentially-offset from one another.

8. The system of claim 1, further comprising a computing system configured to generate a photoacoustic image based at least partially upon the sound.

9. The system of claim 8, wherein the computing system is further configured to determine a spectral response based at least partially upon the photoacoustic image, wherein the spectral response comprises the amplitude of the sound at the different wavelengths of the light, wherein the spectral response being greater than a spectral response threshold indicates an amount of myelin in the nerve is greater than a predetermined myelin threshold, and wherein the spectral response being less than the spectral response threshold indicates the amount of the myelin is less than the predetermined myelin threshold.

10. The system of claim 9, wherein the computing system is further configured to determine a nerve condition of the nerve based at least partially upon the spectral response.

11. A method for assessing a nerve in a patient, the method comprising:
creating an incision in the patient to expose the nerve within the patient;
positioning an encasement at least partially around the nerve;
transmitting light through the encasement to the nerve, wherein a first portion of the light shines on a first portion of the nerve, wherein a second portion of the light shines a second portion of the nerve, and wherein a sound is generated in response to the nerve absorbing the light;
measuring the sound using an ultrasound transducer; and
generating a photoacoustic image based at least partially upon the sound.

12. The method of claim 11, wherein the encasement comprises first and second encasement portions that are configured to actuate between an open position and a closed position, wherein the first and second encasement portions in the closed position are configured to secure the nerve within a channel defined by inner surfaces of the first and second encasement portions.

13. The method of claim 11, wherein the light comprises different wavelengths, wherein a number of the different wavelengths is from about 2 to about 50, wherein the different wavelengths range from about 690 nm to about 2200 nm, wherein the nerve absorbs different amounts of the light at the different wavelengths, and wherein an amplitude of the sound depends at least partially upon the different amounts of the light that are absorbed.

14. The method of claim 13, further comprising determining a spectral response based at least partially upon the photoacoustic image, wherein the spectral response comprises the amplitude of the sound at the different wavelengths, wherein the spectral response being greater than a spectral response threshold indicates an amount of myelin in the nerve is greater than a predetermined myelin threshold, and wherein the spectral response being less than the spectral response threshold indicates the amount of the myelin is less than the predetermined myelin threshold.

15. The method of claim 11, further comprising determining a nerve condition of the nerve based at least partially upon the spectral response.

16. A method for assessing a nerve in a patient, the method comprising:
creating an incision in the patient to expose the nerve within the patient;
positioning an encasement at least partially around the nerve, wherein the encasement comprises first and second encasement portions that are configured to actuate between an open position and a closed position, wherein the first and second encasement portions in the closed position are configured to secure the nerve within a channel defined by inner surfaces of the first and second encasement portions;
moving the encasement along the nerve from a first location to a second location;
transmitting light through the encasement to the nerve, wherein the light is transmitted through the encasement when the encasement is at the first location and the second location, wherein a first portion of the light is transmitted through the first encasement portion onto a first circumferential portion of the nerve, wherein a second portion of the light is transmitted through the second encasement portion onto a second circumferential portion of the nerve, wherein the light comprises different wavelengths, wherein a number of the different wavelengths is from about 2 to about 50, wherein the different wavelengths range from about 690 nm to about 2200 nm, wherein a sound is generated in response to the nerve absorbing the light, wherein the nerve absorbs different amounts of the light at the different wavelengths, and wherein an amplitude of the sound depends at least partially upon the different amounts of the light that are absorbed;
measuring the amplitude of the sound using an ultrasound transducer, wherein the ultrasound transducer is coupled to the encasement;
generating a photoacoustic image based at least partially upon the amplitude of the sound;
determining a spectral response based at least partially upon the photoacoustic image, wherein the spectral response comprises the amplitude of the sound at the different wavelengths of the light, wherein the spectral response being greater than a spectral response threshold indicates an amount of myelin in the nerve is greater than a predetermined myelin threshold, and wherein the spectral response being less than the spectral response threshold indicates the amount of the myelin is less than the predetermined myelin threshold; and
determining a nerve condition of the nerve based at least partially upon the spectral response, wherein the nerve condition is determined at the first location, the second location, or both.

17. The method of claim 16, further comprising:
generating a co-registered ultrasound image using the ultrasound transducer;
overlaying the photoacoustic image on the co-registered ultrasound image to produce an overlaid image; and
locating one or more regions of interest in the nerve based at least partially upon the overlaid image, wherein the nerve condition is determined in one or more regions of interest.

18. The method of claim 16, further comprising comparing the spectral response to a plurality of known spectral responses in a database, wherein the database also comprises a plurality of known nerve conditions that correspond to the known spectral responses, and wherein the nerve condition is determined based at least partially upon the comparison.

19. The method of claim 16, further comprising determining a next surgical step based at least partially upon the nerve condition.

20. The method of claim 19, further comprising performing the next surgical step.
